(19) United States
(12) Patent Application Publication
(10) Pub. No.: US 2019/0209659 A1
(43) Pub. Date: Jul. 11, 2019

(54) STABLE LIQUID FIBRINOGEN

(71) Applicant: LABORATOIRE FRANCAIS DU FRACTIONNEMENT ET DES BIOTECHNOLOGIES, LES ULIS (FR)

(72) Inventors: Damien BATAILLE, ORMOY (FR); Michel TELLIER, EAUBONNE (FR)

(73) Assignee: LABORATOIRE FRANCAIS DU FRACTIONNEMENT ET DES BIOTECHNOLOGIES, LES ULIS (FR)

(21) Appl. No.: 16/315,388

(22) PCT Filed: Jul. 6, 2017
(86) PCT No.: PCT/FR2017/051852

§ 371 Date: Jan. 29, 2019

(30) Foreign Application Priority Data
Jul. 6, 2016 (FR) 16 56479
Jul. 28, 2016 (EP) 16305984.3
Jul. 28, 2016 (EP) 16305985.0

(51) Int. Cl. A61K 38/36; A61K 9/00; A61K 47/18; A61K 47/12

(52) U.S. Cl. CPC A61K 38/363; A61K 47/12; A61K 47/183; A61K 9/0019

(57) ABSTRACT

Disclosed is a composition including stable fibrinogen in liquid form.

### FIELD OF THE INVENTION

This invention relates to a composition comprising stable fibrinogen in liquid form, useful in therapy.

### CONTEXT OF THE INVENTION

Fibrinogen is an essential protein of blood coagulation, because the polymerisation thereof into insoluble fibrin formed at the end of the cascade of reactions that govern coagulation, results in the formation of a clot that seals off the vascular breach, responsible for bleeding.

The setting in place of the clot is thus essential in order to ensure the stopping of the bleeding. In addition, the fibrin formed on the wound forms a fibrillar network which ensures tissue repair, therefore cicatrisation.

Many pathologies are currently treated intravenously by using compositions that contain fibrinogen. Mention can be made for example of constitutional hypofibrinogenemias, dysfibrinogenemias or afibrinogenemias in patients having a spontaneous or post-traumatic bleeding, the additional treatment in caring for severe uncontrolled bleeding in the framework of an acquired hypofibrinogenemia, etc.

For the treatment of certain pathologies, using compositions that contain fibrinogen that is stable in storage and ready to use can be particularly advantageous. This form of administration offers indeed more flexibility and rapidity in administration for practitioners, improving the urgent caring of bleeding patients. For this purpose, compositions containing freeze-dried fibrinogen, stable in storage and suitable for rapid constitution have been developed. However, the reconstitution of such freeze-dried compositions requires a few minutes. In addition, the reconstitution must be carried out gently in order to allow for the complete dissolution of the lyophilisate, guaranteeing the concentration of the product, and this without forming foam, cloudiness or a deposit that would render the composition difficult or impossible to administer.

Using such freeze-dried products is therefore not optimal in a context of intra or prehospital emergency medicine where each minute counts for treating the bleeding.

To date, compositions comprising fibrinogen are marketed in freeze-dried form to be reconstituted and do not provide full satisfaction in terms of liquid stability in particular.

In particular, Chabbat et al (Thrombosis Research, vol. 76, No. 6, 525-533, 1994), describes a fibrinogen composition comprising NaCl, aminocaproic acid, glycine, arginine and aprotinin. Aprotinin, natural competitive inhibitor of serine proteases, is however unfavourable for patients during intravenous administration, on the one hand from the point of view of its animal origin (preparation from bovine lung) with a non-negligible risk of anaphylactic shock, and on the other hand with regards to its anti-coagulant activity that can aggravate the coagulation defect of the patient to be treated.

Application WO0021568 describes compositions comprising fibrinogen, calcium ions, an immunostimulator, and anti-fibrinolytics, for their use as fibrin sealant. However such compositions cannot be administered intravenously to patients.

Application EP1648496 of the applicant, describes compositions comprising fibrinogen, stabilised by the adding of arginine, trisodium citrate, leucine/isoleucine and glycine and/or lysine, allowing for the stabilisation and the solubilisation of the lyophilisate. Such a composition is not however entirely suited for a liquid formulation of fibrinogen.

In this context, the needs for liquid compositions comprising fibrinogen that are easy to use persist.

The Applicant has developed a new composition comprising fibrinogen, preferably human fibrinogen, which is stable in the liquid state.

The invention therefore relates to a composition comprising stable fibrinogen in liquid form.

### SUMMARY OF THE INVENTION

Surprisingly and advantageously, the Applicant revealed that it is possible to obtain compositions comprising fibrinogen that are particularly stable over time in liquid form, using a limited number and minimum quantities of excipients.

### BRIEF DESCRIPTION OF THE FIGURES

FIG. 1A shows the predicted secondary structure of an aptamer of SEQ ID NO: 1. The nucleotides belonging to the base sequence (SEQ ID NO: 66) are highlighted in grey.

FIG. 2A shows the predicted secondary structure of an aptamer of SEQ ID NO: 58. The nucleotides belonging to the base sequence (SEQ ID NO: 67) are highlighted in grey. The framed loop corresponds to the region of the aptamer comprising the consensus group of formula (III).

FIG. 2B shows the alignments of the base sequences of SEQ ID NO: 67-74. The framed portions of the sequences include the consensus group of formula (III).

FIGS. 3-4 show the binding properties of certain aptamers directed against a human fibrinogen obtained by the method of the invention.

FIG. 3A shows the SPR (surface plasmon resonance) binding curves of a human plasma fibrinogen present at a concentration from 125 nM to 1000 nM on the SEQ ID NO: 66 (the base sequence of SEQ ID NO: 1) immobilised on a chip. Each solution of human plasma fibrinogen was injected at pH 6.3 in such a way that a complex was formed in a dose-dependent manner as shown by the increase in the signals depending on the concentration of fibrinogen. The injection of a buffer solution at pH 6.3 comprising 0.5 M of NaCl did not significantly induce the elution of the human plasma fibrinogen. The fibrinogen was then released from the complex by an elution buffer at pH 7.40. The solid support was then regenerated by injection of a solution of NaOH 50 mM. X-axis: time in s. Y-axis: SPR response in arbitrary unit.

FIG. 3B shows the SPR binding curves of a transgenic fibrinogen present at a concentration from 125 nM to 1000 nM on the SEQ ID NO: 66 (the base sequence of SEQ ID NO: 1) immobilised on a chip. Each solution of transgenic fibrinogen was injected at pH 6.3 in such a way that a complex was formed in a dose-dependent manner as shown by the increase in the signals depending on the concentration of fibrinogen. The injection of a buffer solution at pH 6.3 comprising NaCl 0.5 M did not significantly induce the elution of the transgenic fibrinogen. The fibrinogen was then released from the complex by an elution buffer at pH 7.40. The solid support was then regenerated by injection of a solution of NaOH 50 mM. X-axis: time in s. Y-axis: SPR response in arbitrary unit.

FIG. 3C shows the SPR binding curves of a human plasma fibrinogen present at a concentration from 125 nM to 1000 nM on the SEQ ID NO: 67 (the base sequence of SEQ ID NO: 58) immobilised on a chip. Each solution of human plasma fibrinogen was injected at pH 6.3 in such a way that a complex was formed in a dose-dependent manner as shown by the increase in the signals depending on the concentration of fibrinogen. The injection of a buffer solution at pH 6.3 comprising NaCl 1M did not significantly induce the elution of the human plasma fibrinogen. The fibrinogen was then released from the complex by an elution buffer at pH 7.40 and containing MgCl₂ 2 M. The solid support was then regenerated by injection of a solution of NaOH 50 mM. X-axis: time in s. Y-axis: SPR response in arbitrary unit.

FIG. 3D shows the SPR binding curves of a transgenic fibrinogen present at a concentration from 125 nM to 1000 nM on the SEQ ID NO: 67 (the base sequence of SEQ ID NO: 58) immobilised on a chip. Each solution of transgenic fibrinogen was injected at pH 6.3 in such a way that a complex was formed in a dose-dependent manner as shown by the increase in the signals depending on the concentration of fibrinogen. The injection of a buffer solution at pH 6.3 comprising NaCl 1M did not considerably induce the elution of the human plasma fibrinogen. The fibrinogen was then released from the complex by an elution buffer at pH 7.40 and containing MgCl₂ 2 M. The solid support was then regenerated by injection of a solution of NaOH 50 mM. X-axis: time in s. Y-axis: SPR response in arbitrary unit.

FIG. 4A shows SPR sensorgrams showing the dependency of the bond of the fibrinogen with the immobilised aptamer of SEQ ID NO: 66 (the base sequence of SEQ ID NO: 1) with respect to the pH. The plasma fibrinogen is injected at different pH, after the injection of a sample, a migration buffer at pH 6.30 is sent into the flow cell at each test. The highest binding level is obtained at a pH of 6.30. The binding level decreases when the pH increases. X-axis: time in s. Y-axis: SPR response in arbitrary unit.

FIG. 4B shows SPR sensorgrams showing the dependency with respect to the pH of the binding affinity of the aptamer of SEQ ID NO: 67 (the base sequence of SEQ ID NO: 58) with the human plasma fibrinogen. No binding is observed for a pH higher than 6.8. X-axis: time in s. Y-axis: SPR response in arbitrary unit.

FIG. 4C shows the binding curve of a human plasma fibrinogen (sensorgram) for aptamers of SEQ ID NO: 60 and SEQ ID NO: 65 (belonging to the second subgroup of aptamers of the invention) immobilised on a chip, obtained via the SPR technology. Purified human plasma fibrinogen (250 nM) was injected at pH 6.3, in such a way that a complex was formed as shown by the increase in the signal. The injection of a buffer solution at pH 6.3 comprising NaCl 0.5 M did not significantly induce the elution of the human plasma fibrinogen. The solid support was then regenerated by injection of a solution of NaOH 50 mM. X-axis: time in s. Y-axis: SPR response in arbitrary unit.

FIG. 5A shows the chromatographic profile of the purification of the fibrinogen coming from the plasma on an affinity support whereon an aptamer of SEQ ID NO: 66 is grafted. Y-axis: absorbance at 280 nm. X-axis: in mL.

FIG. 5B shows the photo of the electrophoresis gels after a colouration with coomassie blue in non-reduced conditions. From left to right; 1: the plasma; 2: the fraction coming from the plasma that was not retained on the stationary phase, 3: elution fraction containing fibrinogen obtained by chromatography of the plasma, 4: fraction obtained after regeneration of the stationary support, and 5: molecular weight markers. The purity of the elution fraction of the fibrinogen was higher than 95% with respect to the total quantity of proteins contained in the fraction. The affinity support used in the chromatography was grafted with aptamers of SEQ ID NO: 66.

FIG. 6A shows the chromatographic profile of the purification of fibrinogen coming from a plasma on an affinity support whereon an aptamer of SEQ ID NO: 67 is grafted. Y-axis: absorbance at 280 nm. X-axis: in mL.

FIG. 6B shows the photo of the electrophoresis gels after a colouration with coomassie blue in non-reduced conditions. From left to right; 1: the plasma; 2: the fraction coming from the plasma that was not retained on the stationary phase, 3: fraction obtained after washing of the stationary support, 4: elution fraction containing fibrinogen obtained by chromatography of the plasma, and 5: molecular weight markers. The purity of the elution fraction of the fibrinogen was at least 95% with respect to the total quantity of proteins contained in the fraction. The affinity support used in the chromatography was grafted with aptamers of SEQ ID NO: 67.

FIG. 7A shows the chromatographic profile obtained for the purification of semi-purified fibrinogen on an affinity support whereon an aptamer of SEQ ID NO: 66 is grafted. Y-axis: absorbance at 280 nm. X-axis: in mL.

FIG. 7B shows the chromatographic profile obtained for the purification of semi-purified fibrinogen on an affinity support whereon an aptamer of SEQ ID NO: 67 is grafted. Y-axis: absorbance at 280 nm. X-axis: in mL.

FIG. 7C shows the analysis of the fractions via SDS-PAGE in reduced and non-reduced conditions, with a colouration with AgNO₃, of the elution fractions obtained by purification of fibrinogen intermediate on the affinity supports. Track 1: molecular weight standard, Track 2: fibrinogen intermediate (starting material), Track 3: Elution fraction obtained with the affinity support No. 1 (aptamers of SEQ ID NO: 66), Track 4: Elution fraction obtained with the affinity support No. 1 (aptamers of SEQ ID NO: 67).

FIG. 7D shows the analysis of the fractions via SDS-PAGE in reduced and non-reduced conditions, with a colouration with coomassie brilliant blue, of the elution fractions obtained by purification of fibrinogen intermediate on the affinity supports. Track 1: molecular weight standard, Tracks 2 and 3: fibrinogen intermediate (starting material), Track 4 and 5: Elution fraction obtained with the affinity support No. 1 (aptamers of SEQ ID NO: 66), Tracks 6 and 7: Elution fraction obtained with the affinity support No. 1 (aptamers of SEQ ID NO: 67). NR: non-reduced. R: reduced.

FIG. 8 shows the SELEX protocol used to identify aptamers directed against the human fibrinogen.

FIG. 9 shows the competitive binding of an aptamer of SEQ ID NO: 66 immobilised with a fibrinogen injected in the presence of variants of aptamers. Here, the higher the affinity of the variant for the fibrinogen is (in relation to the aptamer of SEQ ID NO: 66), the lower the response during the injection of the variant/fibrinogen mixture is. The variants of SEQ ID NO: 66 comprising one of the following deletion combinations (i) 1/2, (ii) 19/20/21, (iii) 18/19/20/21, (iv) 15/16/19/20 and (v) 14/15/16/20/21/22 have a strong affinity for the fibrinogen.

FIG. 10A shows the binding curves of human transgenic and plasma fibrinogen (sensorgram) for an aptamer coming from Base Pair Biotechnologies (reference 6F01 oligo#370) immobilised on a sensor chip, obtained via the SPR technology. The human transgenic and plasma fibrinogen (1000 nM) was injected at pH 7.40 using a buffer recommended by Base Pair Biotechnologies. Very low binding levels were observed for the human transgenic and plasma fibrinogen. The solid support was then regenerated by injection of a solution of NaOH 50 mM. X-axis: time in s. Y-axis: response in arbitrary unit.

FIG. 10B shows the binding curves of human plasma fibrinogen (sensorgram) for 3 aptamers (aptamers 85A, 121A and 121B) described in the additional data of Li et al. (J Am Chem Soc, 2008, 130 (38):12636-12638) immobilised on a sensor chip, obtained via the SPR technology. The human plasma fibrinogen (1000 nM) was injected at pH 7.40, as recommended by Li et al. Very low binding levels were observed for the human transgenic and plasma fibrinogen. The solid support was then regenerated by injection of a solution of NaOH 50 mM. X-axis: time in s. Y-axis: response in arbitrary unit.

### Notes:

The buffer MBS designates 50 mM of MOPS/150 mM of NaCl.

The buffer of NaCl 1M MBS designates 50 mM of MOPS/1 M of NaCl.

The buffer M5 MBS designates: 50 mM of MOPS pH 6.30/150 mM of NaCl/5 mM of MgCl₂.

The buffer 0.5M M5 MBS designates 50 mM of MOPS pH 6.30/0.5 M of NaCl/5 mM of MgCl₂.

### DETAILED DESCRIPTION OF THE INVENTION

Fibrinogen is a protein constituted of a dimer of three polypeptide chains named alpha, beta and gamma. Fibrinogen is therefore a dimer and each monomer comprised three chains (alpha, beta, gamma). The main form of fibrinogen has a molecular weight (MW) of 340 kDa. Fibrinogen is formed from two identical subunits connected by disulphide bridges, giving to the molecule a form of fibre comprising 3 globules: one central (domain E) and two distal (domains D). The entire molecule contains 2,964 amino acids: 610 amino acids for the alpha chain (α), 461 amino acids for the beta chain (β), and 411 amino acids for the gamma chain (γ). Fibrinogen intervenes in primary haemostasis as well as in coagulation. It is most often prescribed for treating complications associated with constitutional or severe afibrinogenemia and haemorrhagic syndromes or risks of bleeding associated with a hypofibrinogenemia.

The term “stable” corresponds to the physical and/or chemical stability of the composition comprising the fibrinogen. The term “physical stability” refers to the reduction or the absence of the formation of insoluble or soluble aggregates of the dimer, oligomer or polymer forms of the fibrinogen, and to the reduction or absence of the formation of precipitate, as well as to the reduction or the absence of any structural denaturation of the molecule.

The term “chemical stability” refers to the reduction or absence of any chemical modification of the composition comprising the fibrinogen during the storage, in the liquid state, in accelerated conditions.

A stability test can be carried out in different conditions of temperature, humidity, and light. Preferably, in the framework of this invention, the stability test can last at least 1 week, preferably at least 1 month, preferably at least 2 months, preferably at least 3 months, preferably at least 4 months, preferably at least 5 months, more preferably at least 6 months. Typically, the measurement of the stability parameters, such as defined hereinafter, take place
before the stability testing of a composition comprising fibrinogen; this is then referred to as initial rate; and
during or after said stability test,
   with the understanding that said stability test can last at least 1 week, preferably at least 1 month, preferably at least 2 months, preferably at least 3 months, preferably at least 4 months, preferably at least 5 months, more preferably at least 6 months.

The stability of a composition comprising fibrinogen can be evaluated via a visual inspection using in particular a European pharmacopeia inspection machine (opalescence, formation of particles), by measuring the turbidity using a spectrophotometer that measures the absorbance or optical density at 400 nm, making it possible to measure the particles in solution with sizes between about 1 nm and 1 μm.

In an embodiment, the stability of the composition comprising fibrinogen is defined by the measurement of the rate of the monomers retained during the stability test using the High Pressure Size Exclusion Chromatography (HPSEC) or Dynamic Light Scattering (DLS) method. These methods are well known to those skilled in the art.

A fibrinogen composition is advantageously considered to be stable if the quantity of fibrinogen monomers retained during stability testing is higher than 50%, preferably higher than 60%, preferably higher than 70%, preferably higher than 80%, preferably higher than 90%, preferably higher than 95% of the initial rate of fibrinogen monomers.

More preferably, the quantity of fibrinogen monomers retained during the stability test is higher than 70% of the initial rate of fibrinogen monomers.

The term “initial rate of fibrinogen monomers” means the rate of monomers observed before stability testing. Typically, the quantity of fibrinogen monomers is measured before stability testing and during or after said stability test.

Alternatively, a fibrinogen composition is considered to be stable if the variation in the quantity of fibrinogen monomers during the stability test is lower than 20%, preferably lower than 10%, preferably lower than 5%, preferably lower than 1%.

In another embodiment, the stability of the composition comprising fibrinogen is defined by the measurement of the rate of fibrinogen polymers formed during stability testing by using HPSEC. Fibrinogen polymers are polymers comprising at least 2 alpha polypeptide chains, 2 beta polypeptide chains and 2 gamma polypeptide chains of fibrinogen. This term also includes fibrinogen trimers.

A fibrinogen composition is advantageously considered to be stable if the quantity of fibrinogen polymers formed during stability testing is lower than 50%, preferably lower than 40%, preferably lower than 30%, preferably lower than 20%, preferably lower than 10% in relation to the initial rate of fibrinogen polymers. Typically, the initial rate of fibrinogen polymers corresponds to all of the polymer forms (trimers and more) of the fibrinogen before stability testing.

More preferably, the quantity of fibrinogen polymers formed during stability testing is lower than 30%. Typically, the quantity of fibrinogen polymers is measured before stability testing and during or after said stability test.

Alternatively, a fibrinogen composition is considered to be stable if the variation in the quantity of fibrinogen polymers during the stability test is lower than 20%, preferably lower than 10%, preferably lower than 5%, preferably lower than 1%.

In another embodiment, the stability of the composition comprising fibrinogen is evaluated by measuring the coagulable activity of the fibrinogen with respect to the antigen measurement of the fibrinogen (also called specific activity). Particularly advantageously, the stable fibrinogen composition has a coagulable fibrinogen/fibrinogen antigen ratio higher than 0.5; preferably higher than 0.6; higher than 0.7; higher than 0.8; higher than 0.9; even more preferably equal to about 1.0. Even more advantageously, a fibrinogen composition is considered to be stable if the ratio of its coagulable activity to its antigen activity at the end of the test is higher than at least 60%, preferably at least 70%, 80%, 90%, 95%, 98% or 99% of the initial value of this ratio (its value before the stability test).

The term “coagulable fibrinogen” means the measurement of the functional fibrinogen via a coagulation technique, determined according to the method of von Clauss. The coagulable activity is expressed in g/L of fibrinogen solution. This technique is known to those skilled in the art who can refer to the publication Von Clauss, A. (1957) Gerinnungsphysiologische schnellmethode zur bestimmung des fibrinogens. Acta Haematologica, 17, 237-246.

The term “antigen fibrinogen” means the quantity of fibrinogen, whether it is active or not, measured by nephelometric method. The quantity of antigen fibrinogen is expressed in g/L.

The stability of the composition comprising fibrinogen is also evaluated by the measurement in SDS PAGE of the retaining of the alpha, beta and gamma chains of fibrinogen, preferably before and after a stability test as defined in the framework of this invention. Thus, a fibrinogen composition is advantageously considered to be stable if:
all of the alpha chains are retained at at least 50%, preferably at least 60%, preferably at least 70%, preferably at least 80%, preferably at least 90%; more preferably retained at about 100%, and/or
all of the beta chains are retained at at least 50%, preferably at least 60%, preferably at least 70%, preferably at least 80%, preferably at least 90%; more preferably retained at about 100%, and/or
all of the gamma chains are retained at at least 50%, preferably at least 60%, preferably at least 70%, preferably at least 80%, preferably at least 90%; more preferably retained at about 100%.

In particular, the fibrinogen composition is considered to be stable if the profiles of the alpha and gamma chains are retained at the end of the stability test. The term retaining of the profiles of the alpha and gamma chains means the retaining of the distribution of the different forms of the alpha and gamma chains.

Thus the profile of the alpha chains is considered to be retained during the stability test if the distribution of the forms Aα1, Aα2 and Aα3 of the alpha chain is retained between the initial measurement taken before stability testing and that taken at the end of the period of stability testing. A fibrinogen composition can be considered to be stable if the extent of the form Aα1 (expressed as a percentage of all of the forms of the alpha chains) after stability testing corresponds to at least 50%, more preferably to at least 60%, 70%, 80% or 90% of the extent of the form Aα1 (expressed as a percentage of all of the forms of the alpha chains) before stability testing.

Thus the profile of the gamma chains is considered to be retained during the stability test if the distribution of the forms γ′ and γ of the gamma chain is retained between the initial measurement taken before stability testing and that taken at the end of the period of stability testing.

The stability of the composition comprising fibrinogen is also defined by the measurement of the turbidity using UV spectrophotometry at 400 nm. Indeed, the turbidity reflects the quantity of matter that is clouding the solution. A fibrinogen composition is advantageously considered to be stable if the turbidity measured after the stability test as defined in this invention is comparable to the turbidity measured before stability.

Advantageously, the turbidity measured after stability testing corresponds to lower than 130%, lower than 120%, lower than 110%; advantageously corresponds to 100% of the turbidity measured before stability.

In an advantageous embodiment of the invention, the composition comprising fibrinogen is stable for at least 1 month, at least 2 months, at least 3 months, at least 4 months, at least 5 months, at least 6 months at 4° C.

The term “composition according to the invention” intends to designate the composition comprising fibrinogen, said composition being stable in liquid form. Preferably, said composition is constituted of fibrinogen, arginine and citrate.

The term “fibrinogen composition in liquid form” means a composition comprising fibrinogen in solution, preferably that has not been subjected to a step of freeze-drying, desiccation, dehydration or drying, and which therefore does not need to be reconstituted before use.

An object of the invention is therefore a pharmaceutical composition comprising stable fibrinogen in liquid form that is not reconstituted after freeze-drying. Another object of the invention is a pharmaceutical composition comprising fibrinogen and one or more pharmaceutically acceptable excipients, stable in liquid form.

The term “pharmaceutically acceptable excipient” corresponds to any excipient that can advantageously be used for the formulation of human proteins, in particular with substances chosen from salts, amino acids, sugars, surfactants or any other excipient.

The pharmaceutically acceptable excipients of the invention exclude in particular isoleucine, glycine, and NaCl.

Advantageously, the pharmaceutically acceptable excipients according to the invention include arginine and/or citrate. The Applicant revealed that it was possible to obtain compositions that are particularly stable over time in liquid form comprising fibrinogen, arginine and citrate. Such a composition is optimal, because it limits the number of excipients and therefore the risk of side effects due to the components of the formulation while still allowing for storage in liquid form of the ready-to-use composition.

Thus, in a preferred embodiment, the invention relates to a composition comprising, preferably constituted, of fibrinogen, of arginine, and of citrate, and which is stable in liquid form.

Preferably, said fibrinogen is a human fibrinogen.

According to the invention, several sources of raw material containing fibrinogen can be used. The fibrinogen composition can thus come from blood plasma also called plasma fractions, supernatant of cell culture or from the milk of transgenic animals.

In a preferred embodiment, the composition of the invention has not been subjected to any prior step of freeze-drying, desiccation, dehydration or drying.

In a preferred embodiment, the composition of the invention has not been subjected to any prior step of reconstituting a lyophilisate.

In a particular embodiment, the composition according to the invention is a plasma fraction, more preferably a plasma fraction obtained from prepurified blood plasma.

The term “plasma fraction obtained from prepurified blood plasma” means any portion or subportion of human blood plasma, having been subjected to one or several steps of purification. Said plasma fractions thus include plasma cryosupernatant precipitate, plasma cryoprecipitate (re-suspended), the fraction I obtained by ethanol fractionation (according to the Cohn method or the Kistler & Nitschmann method), chromatography eluates and unabsorbed fractions of chromatography columns, including multi-column chromatographies, and filtrates.

In a preferred embodiment of the invention, the composition according to the invention comes from a chromatography eluate or from an unabsorbed chromatography column fraction, including multi-column chromatography.

In an even more preferred embodiment of the invention, the composition according to the invention comes from a chromatography eluate or from an unabsorbed chromatography column fraction, except multi-column chromatography.

Thus, in a preferred embodiment of the invention, the composition according to the invention comes from a plasma fraction obtained from cryosupernatant or from re-suspended cryoprecipitate.

According to the invention, the “plasma cryosupernatant precipitate”, or “cryosupernatant”, corresponds to the liquid phase obtained after defrosting of frozen plasma (cryoprecipitation). In particular, the cryosupernatant can be obtained by defrosting blood plasma at a temperature between −10° C. and −40° C., then slow defrosting at a temperature between 0° C. and +6° C., preferably between 0° C. and +1° C., followed by a centrifugation of the defrosted plasma in order to separate the cryoprecipitate and the cryosupernatant. The cryoprecipitate is a fibrinogen, fibronectin, von Willebrand factor and factor VIII concentrate, while the cryosupernatant contains complement factors, vitamin K-dependent factors such as protein C, protein S, protein Z, factor II, factor VII, factor IX and factor X, fibrinogen, immunoglobulins and albumin.

In an advantageous embodiment of the invention, the composition according to the invention may be obtained according to the method described by the Applicant in application EP1739093 or in application WO2015/136217.

In another particular embodiment of the invention, the composition according to the invention comes from the milk of transgenic animals, for example obtained according to the method described in WO00/17234 or in WO00/17239.

In an embodiment, the composition according to the invention comes from plasma that has not been depleted in proteins beforehand such as immunoglobulins or albumin.

In an embodiment, the composition of the invention is able to be obtained by the method comprising the following steps:
a step of purification by affinity chromatography;
at least one step of biological security; and
a step of formulation in liquid form.

In a particular embodiment of the invention, the step of purification by affinity chromatography is carried out by affinity chromatography using affinity ligands chosen from antibodies, fragments of antibodies, derivatives of antibodies or chemical ligands such as peptides, peptide mimetics, peptoids, nanofitins or oligonucleotide ligands such as aptamers.

In a particular embodiment of the invention, the step of purification by affinity chromatography is carried out by affinity chromatography using affinity ligands being aptamers.

The applicant has conducted his own research and has identified a new family of aptamers, directed against fibrinogen. This new family of aptamers was identified internally by a

SELEX method designed by the applicant. These aptamers are shown to bind specifically with both human transgenic and plasma fibrinogen, independently of the state of glycosylation of the protein. The aptamers identified by the applicant have unique properties in terms of binding. In particular, the aptamers used according to the invention bind to the fibrinogen in a pH-dependent manner. Note that they have an increased binding affinity for fibrinogen at a slightly acidic pH such as a pH of about 6.3, in relation to a pH higher than 7.0, such as 7.4. These properties are particularly suitable for use in affinity chromatography because the formation of the complex between the protein to be purified, namely fibrinogen, and the aptamer, and the later release of the protein from the complex can be controlled by modifying the pH of the elution buffer. In particular, the release of the protein from the complex can be carried out in moderate conditions of elution, which are not able to alter the properties of the protein.

•Aptamers used according to the invention

The aptamers are used according to the invention as affinity ligands in the purification of the fibrinogen, for example by chromatography. The aptamers used according to the invention are therefore directed against fibrinogen, namely able to bind specifically with the fibrinogen. The aptamers used according to the invention bind to the fibrinogen in a pH-dependent manner.

Preferably, the aptamers used according to the invention do not bind to the fibrinogen at a pH higher than 7.0 and bind to the fibrinogen at an acid pH, for example at a pH value chosen from 6.0 to 6.6, such as pH 6.3±0.1.

As is meant here, an “aptamer” (also called nucleic aptamer) designates a synthetic single-stranded polynucleotide typically having a length from 20 to 150 nucleotides and capable of binding with great affinity to a target molecule. The aptamers are characterised by one or more three-dimensional conformations which can play a key role at the level of their interactions with their target molecule. Consequently, the aptamer used according to the invention is capable of forming a complex with the fibrinogen. The interactions between an aptamer and its target molecule can encompass electrostatic interactions, hydrogen bonds, and a complementarity in the form of an aromatic stack.

“An aptamer that binds specifically to its target molecule” means that the aptamer has great affinity for the target molecule. The dissociation constant (Kd) of an aptamer for its target molecule is typically from 10-6 to 10-12 M. The term “binds specifically” is used here to indicate that the aptamer has the capacity to recognise and to interact specifically with its target molecule, while still having a detectable reactivity that is relatively low with other molecules that may be present in the sample. Preferably, the aptamer binds specifically to its target molecule if its affinity is significantly higher for the target molecule, than for other molecules, including molecules that structurally close to the target molecule. For example, an aptamer can be capable of specifically binding to a human protein while still having a lower affinity for an equivalent of said human protein.

As is meant here, “an aptamer that has a lower affinity for a given molecule than for its target molecule” or “an aptamer which is specific to its target molecule in relation to a given molecule” means that the Kd of the aptamer for said given molecule is at least 5 times, more preferably at least 10, 20, 30, 40, 50, 100, 200, 500, or 1000 times higher than the Kd of said aptamer for the target molecule.

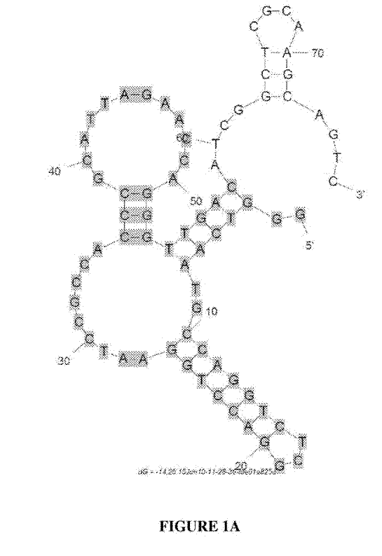

The aptamers can be a deoxyribonucleic acid (DNA) or a ribonucleic acid (RNA). The aptamers can include one or several chemically modified nucleotides. Chemically modified nucleotides encompass, without being limited thereto, 2′-amino or 2′-fluoro-nucleotides, 2′-ribopurine, phosphoramidite, a locked nucleic acid (LNA), nucleotides modified by boronic acid, 5-iodo- or 5-bromo-uracil, and deoxyuridine 5-modified such as benzyl-dU, isobutyl-dU, and napthyl-dU. For deoxyuridine 5-modified, reference can be made to Rohloff et al., Molecular Therapy-Nucleic acids, 2014, 3, e201 (see FIG. 1 page 4), of which the description is incorporated here as a reference. In certain embodiments, the aptamer used according to the invention is devoid of any nucleotide modified by boronic acid, in particular those taught in WO2010/019847. In certain other embodiments, the aptamer used according to the invention is devoid of any deoxyuridine 5-modified. In certain embodiments, the aptamer can include a modified nucleotide at the level of its end 3′ and/or of its end 5′ only (i.e. the first nucleotide and/or the last nucleotide of the aptamer is/are the only chemically-modified nucleotide or nucleotides). Preferably, said modified nucleotide can allow for the grafting of the aptamer on a solid support, or the coupling of said aptamer to any group of interest (for example, useful for a detection or an immobilisation).

When the sequence of the aptamer is identified, the aptamer can be prepared by any routine method known to those skilled in the art, namely by a chemical oligonucleotide synthesis, for example in the solid phase.

As is meant here, “an aptamer directed towards the fibrinogen”, “an aptamer directed against the fibrinogen” or “an anti-fibrinogen aptamer” designates a synthetic single-stranded polynucleotide that binds specifically to the fibrinogen.

As is meant here, the term “fibrinogen” designates any protein that has the amino acid sequence of the wild-type fibrinogen and variants of the latter, independently of the state of glycosylation. The term “fibrinogen” encompasses any isoform and gene variant of the fibrinogen, as well as any glycosylated form, any non-glycosylated form or any post-translational form of the fibrinogen.

As is meant here, a variant of the wild-type fibrinogen designates a protein having a sequence identity of at least 80%, more preferably a sequence identity of at least 85%, 90%, or 95%, with said wild-type fibrinogen and which has a biological activity similar to that of said wild-type fibrinogen. For example, the coagulation activity of the fibrinogen can be measured by the van Clauss coagulation method. The variant of fibrinogen can have an increased or reduced biological activity in relation to the corresponding wild-type fibrinogen.

In certain embodiments, the fibrinogen designates a protein having the amino acid sequence of the wild-type human fibrinogen or a variant of the latter. Said fibrinogen can be human plasma fibrinogen, human recombinant or transgenic fibrinogen. In certain embodiments, the aptamer used according to the invention is capable of binding to the human fibrinogen, independently of its glycosylation. For example, an aptamer of the invention can be capable of binding specifically to the human plasma fibrinogen and to the recombinant human fibrinogen, for example the recombinant fibrinogen obtained from a multicellular transgenic organism or the recombinant fibrinogen obtained from a recombinant host cell.

The aptamers used according to the invention can be capable of binding specifically to the fibrinogen at a slightly acidic pH, for example at pH 6.3.

Preferably, the aptamer used according to the invention has a dissociation constant (Kd) for the human plasma fibrinogen or for the human transgenic fibrinogen of at most 10-6 M. Typically, the Kd of the aptamers used according to the invention for the human fibrinogen can be from 1.10-12 M to 1.10-6 M at a pH of about 6.3. The Kd is more preferably determined by a surface plasmon resonance (SPR) test wherein the aptamer is immobilised on the biosensor chip and the fibrinogen is sent over the immobilised aptamers, at a pH of interest, and at various concentrations, in flow conditions that lead to the measurements of kon and of koff and therefore of the Kd. Reference can be made to the protocol provided in the example 3.

In certain embodiments, the aptamer used according to the invention is specific of human fibrinogen, in relation to non-human fibrinogen.

In certain other embodiments, the aptamer used according to the invention is specific of human fibrinogen in relation to other proteins present in the plasma, such as coagulation factors. Preferably, the aptamer used according to the invention binds specifically to the fibrinogen in relation to factor FII, FXI or XIII. In additional or alternative embodiments, the aptamer used according to the invention binds specifically to the fibrinogen in relation to a fibronectin. In additional or alternative embodiments, the aptamer used according to the invention binds specifically to the fibrinogen in relation to a plasminogen.

Preferably, the aptamer used according to the invention does not bind to the fibrinogen at a pH of 7.0 or more. The inability of the aptamer used according to the invention to bind to the fibrinogen at a pH of 7.0 and more can typically be determined by SPR, such as described in the example 3. In the protocol of the example 3, an absence of a bond is represented by the fact that the SPR signal remains at the level of the base line after the injection of the fibrinogen into a buffered buffer at a pH of interest.

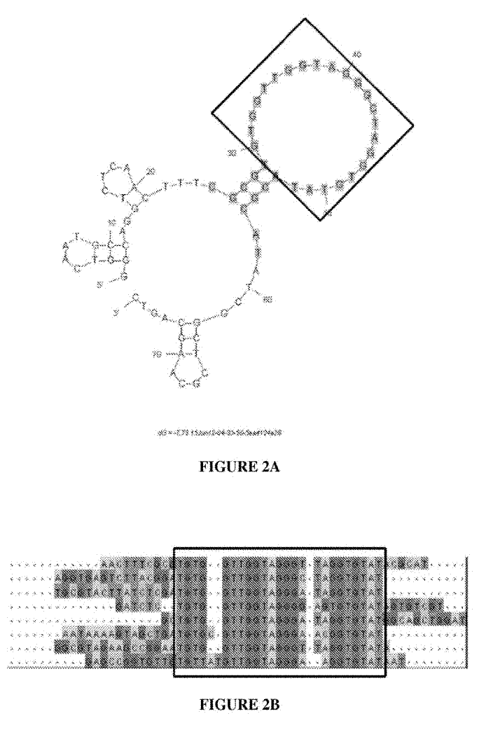

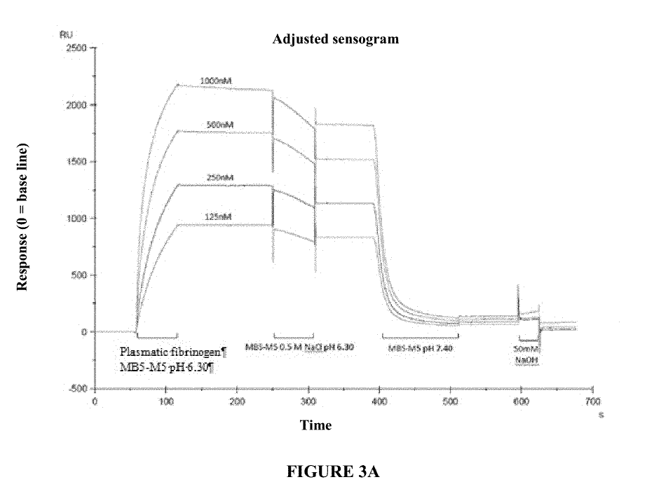

In a certain aspect of the invention, the aptamers can be characterised by the presence of a specific group in their conformation. For example, the aptamers used according to the invention can include a group such as shown in FIG. 1A or in FIG. 2A. Without intending to be bound by any theory, the applicant thinks that the presence of said two-dimensional conformation can be involved in the specific interactions with the fibrinogen.

The presence of said specific conformational group can come from the presence of a specific polynucleotide (called “base polynucleotide” or “base sequence”) within the aptamer sequence. As is meant here, a “base sequence” of a given aptamer typically includes, or designates, the minimum sequence coming from said aptamer that is able to bind to the fibrinogen.

By studying the aptamers identified by his own research, the applicant has identified several base sequences of interest, among which polynucleotides of SEQ ID NO: 66 and of SEQ ID NO: 67.

The applicant has furthermore determined the groups of the consensus sequence in the SEQ ID NO: 58-65. As shown in FIG. 2B, the aptamers of SEQ ID NO: 58-65 include two consensus groups in their sequences, namely GTTGGTAGGG (SEQ ID NO: 77) which is upstream of GGTGTAT (SEQ ID NO: 78). These consensus groups are located in a region of the aptamers that forms a loop shown in FIG. 2A for the aptamer of SEQ ID NO: 58.

Without intending to be bound by any theory, the applicant thinks that this conformational group can play a role in the binding of said aptamers to the fibrinogen.

In a certain aspect, the aptamer used according to the invention is able to bind specifically to the fibrinogen and has one of the following characteristics:
Said aptamer comprises a polynucleotide having a sequence identity of at least 70%, for example of at least 75%, 80%, 85%, 90%, or 95%, with the nucleotide sequence of SEQ ID NO: 66, or
Said aptamer comprises the nucleotide groups GTTGGTAGGG (SEQ ID NO: 77) and GGTGTAT (SEQ ID NO: 78), wherein the group of SEQ ID NO: 77 is more preferably upstream of SEQ ID NO: 78.

In certain embodiments, the aptamer used according to the invention is able to bind specifically to the fibrinogen and has one of the following characteristics:
Said aptamer comprises a polynucleotide having a sequence identity of at least 70% with the nucleotide sequence of SEQ ID NO: 66, or
Said aptamer comprises the nucleotide group of formula (III):

5′-[SEQ ID NO: 79]-[X1]-[SEQ ID NO: 77]-[X2]-[SEQ ID NO: 78]-3′ (III) wherein:
[X2] and [X1] independently designate a nucleotide or an oligonucleotide of a length from 2 to 5 nucleotides, more preferably of a length of 2 or 3 nucleotides,
[SEQ ID NO: 77] is an oligonucleotide of SEQ ID NO: 77 (namely GTTGGTAGGG),
[SEQ ID NO: 78] is an oligonucleotide of SEQ ID NO: 78 (namely GGTGTAT) and
[SEQ ID NO: 79] is an oligonucleotide of SEQ ID NO: 79 (namely TGT).

In a particular embodiment, the aptamer used according to the invention comprises a polynucleotide that:
has a sequence identity of at least 70% with at least one sequence of nucleotides chosen from the group comprising SEQ ID NO: 67, SEQ ID NO: 68, SEQ ID NO: 69, SEQ ID NO: 70, SEQ ID NO: 71, SEQ ID NO: 72, SEQ ID NO: 73 SEQ ID NO: 74, and SEQ ID NO: 95 and
comprises the nucleotide group of formula (III) such as defined hereinabove.

For example, the aptamer used according to the invention can include a polynucleotide that has a sequence identity of at least 70% with SEQ ID NO: 67 and which comprises the nucleotide group of formula (III).

In another aspect, the aptamer used according to the invention is able to bind specifically to the fibrinogen and comprising a polynucleotide having a sequence identity of at least 70% with the SEQ ID NO: 66 or the SEQ ID NO: 67.

As is meant here, a sequence identity of at least 70% encompasses a sequence identity of at least 75%, 80%, 85%, 86%, 87%, 88%, 89%, 90%, 91%, 92%, 93%, 94%, 95%, 96%, 97%, or 98%. The “percentage identity” between two sequences of nucleotides (A) and (B) can be determined by comparing the two sequences aligned in an optimum manner, via a comparison window. Said alignment of the sequences can be carried out by well-known methods, for example, using the Needleman-Wunsch global alignment algorithm. When the alignment is obtained, the percentage identity can be obtained by dividing the total number of aligned identical amino acid residues by the total number of residues contained in the longest sequence between the sequences (A) and (B). The sequence identity is typically determined using a sequence analysis software. To compare two nucleic acid sequences, it is possible to use, for example, the “Emboss needle” tool of an alignment of sequences by pairs in order to provide a EMBL-EBI and available on http://www.ebi.ac.uk/Tools/psa/embossneedle/nucleodite.html using the default settings: (i) Matrix: full DNA, (ii) space opening: 10, (iii) space level: 0.5, (iv) output format: even, (v) terminal space penalty: false, (vi) space opening terminal: 10, (vii) space level terminal: 0.5.

The aptamer used according to the invention typically has a length from 20 to 150 nucleotides, more preferably a length from 30 to 100 nucleotides, for example a length from 25 to 90 nucleotides, a length from 30 to 80 nucleotides or a length from 30 to 60 nucleotides. Consequently, the aptamer used according to the invention can have a length of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 nucleotides.

In a particular embodiment, the aptamer used according to the invention comprises a polynucleotide that differs from a polynucleotide chosen from the group of SEQ ID NO: 66 and of SEQ ID NO: 67 at a rate of 1 to 15 nucleotide modifications, more preferably at a rate of 1 to 10 nucleotide modifications, such as 1, 2, 3, 4, 5, 6, 7, 8, 9, or 10 nucleotide modifications.

As is meant here, a “nucleotide modification” designates the deletion of a nucleotide, the insertion of a nucleotide or the substitution of a nucleotide with another nucleotide in relation to the reference sequence.

The aptamers used according to the invention can also include primers at their ends 3′ and 5′ useful for the amplification thereof by PCR. In certain embodiments, these primer sequences can be incorporated or partially incorporated into the base sequence and can therefore participate in binding interactions with the fibrinogen. In certain other embodiments, these primer sequences are outside of the base sequence and may not play any role whatsoever in the interaction of the aptamer with the fibrinogen. In certain other embodiments, the aptamer is devoid of primer sequences.

In certain alternative or additional embodiments, the aptamer used according to the invention can include a polynucleotide with a length from 2 to 40 nucleotides bonded to the end 5′ and/or to the end 3′ of the base sequence.

In a certain aspect, the aptamer used according to the invention binds specifically to the fibrinogen and responds to the formula (I)

5′-[NUC1]m-[CENTRAL]-[NUC2]n-3′ (I)

wherein
n and m are integers chosen independently from 0 and 1,
[NUC1] is a polynucleotide comprising from 2 to 40 nucleotides, more preferably from 15 to 25 nucleotides
[NUC2] is a polynucleotide comprising from 2 to 40 nucleotides, more preferably from 15 to 25 nucleotides and
[CENTRAL] is a polynucleotide having a sequence identity of at least 70% with a sequence of nucleotides chosen from the group comprising SEQ ID NO: 68, SEQ ID NO: 69 SEQ ID NO: 70, SEQ ID NO: 71, SEQ ID NO: 72, SEQ ID NO: 73, SEQ ID NO: 74, SEQ ID NO: 94 and SEQ ID NO: 95.

When n=m=0, [NUC1] and [NUC2] are absent and the aptamer is constituted of the sequence [CENTRAL]. When n=0 and m=1, [NUC2] is absent and [NUC1] is present, the aptamer therefore responds to the formula (Ia)

5′-[NUC1]-[CENTRAL]-3′,

When n=1 and m=0, [NUC1] is absent and [NUC2] is present, the aptamer therefore responds to the formula (Ib)

5′-[CENTRAL]

In certain embodiments, [NUC1] comprises, or is constituted of a polynucleotide of SEQ ID NO: 75 or of a polynucleotide that differs from SEQ ID NO: 75 at a rate of 1, 2, 3 or 4 nucleotide modifications. In certain other additional embodiments, [NUC2] comprises, or is constituted of a polynucleotide of SEQ ID NO: 76 or of a polynucleotide that differs from SEQ ID NO: 76 at a rate of 1, 2, 3 or 4 nucleotide modifications.

In another aspect, the invention relates to an aptamer directed against the fibrinogen and which has a sequence identity of at least 70%, such as at least 75%, 80%, 85%, 90%, 95%, 96%, 97% or 98% with a sequence of nucleotides chosen from the group comprising SEQ ID NO: 1 to SEQ ID NO: 67. For example, the aptamer used according to the invention can have a sequence identity of at least 70% with a nucleotide sequence chosen from SEQ ID NO: 1, SEQ ID NO: 2, SEQ ID NO: 58, SEQ ID NO: 60, SEQ ID NO: 65, SEQ ID NO: 66 and SEQ ID NO: 67.

The applicant has carried out an in-depth analysis of the sequences and of the possible conformations of the aptamers defined hereinabove. This analysis led to the identification of two subgroups of aptamers, with each subgroup being characterised by specific functional and structural properties.

### —First Subgroup of Aptamers Used According to the Invention

The first subgroup of aptamers encompasses aptamers directed against fibrinogen that comprises a base sequence that has a large sequence identity with the base sequence of SEQ ID NO: 66. This first subgroup of aptamers of SEQ ID NO: 1-57 and the aptamer constituted of the base sequence of SEQ ID NO: 66.

Consequently, the aptamer used according to the invention binds specifically to the fibrinogen and comprises a polynucleotide having a sequence identity of at least 70%, 75%, 80%, 85%, 90%, 92%, 94%, 95%, 96%, 97% or 98% with SEQ ID NO: 66. Preferably, said aptamer has a length from 20 to 110 nucleotides, in particular a length from 25 to 100 nucleotides, such as a length of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99. In particular, the aptamer can have a length from 35 to 65 nucleotides.

In certain embodiments, the aptamer comprises a polynucleotide of SEQ ID NO: 66, or a polynucleotide that has a nucleotide sequence that differs from SEQ ID NO: 66 at a rate of 1 to 16 nucleotide modifications, such as 1, 2, 3, 4, 5, 6, 7, 8, 9, 10, 11, 12, 13, 14 or 15 nucleotide modifications. As mentioned hereinabove, the nucleotide modification or modifications can be of any type. A nucleotide modification can be a deletion of a nucleotide, the insertion of a nucleotide or the substitution/replacing of a nucleotide with another nucleotide.

The alignment of the base sequence of SEQ ID NO: 66 with aptamers of SEQ ID NO: 1-57 has shown that certain nucleotides are not retained among the aptamers that belong to the first subgroup. Said positions encompass the positions 19, 20, 21, 24, 27, 32, 33, 35, 42, 45, 46, 47, 50, 54, 55 and 57, with the numbering designating a numbering of nucleotides in SEQ ID NO: 66.

Other modifications, in particular one or several deletions, can be introduced at positions 1 and 2 of SEQ ID NO: 66 but also in the third rod of the base sequence of SEQ ID NO: 66, as shown in FIG. 1A. In particular, the applicant has introduced one to six deletions into the third rod of the aptamer of SEQ ID NO: 66, in particular at the positions 14-22, without any significant loss of affinity to the fibrinogen in relation to the parent base sequence of SEQ ID NO: 66.

Consequently, the nucleotide modification or modifications in relation to the SEQ ID NO: 66 can be present in one or several of these nucleotide positions. In certain embodiments, the aptamer used according to the invention comprises a polynucleotide that differs from the SEQ ID NO: 66 at a rate from 1 to 20, more preferably from 1 to 14, in particular from 1, 2, 3, 4, 5 or 6 nucleotide modifications at the nucleotide positions chosen from 1, 2, 11-25, 32-35, 42, 45-47, 50 and 54-58, more preferably at the nucleotide positions chosen from 1, 2, 14-22, 24, 27, 32, 33, 35, 42, 45, 46, 47, 50, 54, 55 and 57, the numbering referring to the nucleotide numbering in SEQ ID NO: 66. Preferably, the modification or modifications are one or more nucleotide replacements or deletions.

In certain particular embodiments, said nucleotide modification or modifications appear at the nucleotide positions chosen from 20, 35, 42 and 55. In certain particular embodiments, the aptamer used according to the invention can include a polynucleotide that differs from the SEQ ID NO: 66 at a rate of at most 4 nucleotide modifications that appear more preferably at the positions chosen from 20, 35, 42 and 55, the numbering referring to the nucleotide numbering in SEQ ID NO: 66.

For example, the aptamer used according to the invention can include a polynucleotide of SEQ ID NO: 66, or a polynucleotide that has a nucleotide sequence that differs from the SEQ ID NO: 66 at a rate of 1, 2 or 3 nucleotide modification or modifications, more preferably at a rate of 1, 2 or 3 nucleotide modification or modifications, said nucleotide modification or modifications being in one or more nucleotide positions chosen in the group comprising 19, 20, 21, 24, 27, 32, 33, 35, 42, 45, 46, 47, 50, 54, 55 and 57, the numbering referring to the nucleotide numbering in SEQ ID NO: 66.

In certain other embodiments, the aptamer used according to the invention can include the polynucleotide of SEQ ID NO: 66, or a polynucleotide that has a nucleotide sequence that differs from the SEQ ID NO: 66 at a rate of 1-14 nucleotide deletion or deletions, more preferably at a rate of 1, 2, 3, 4, 5, 6 or 7 nucleotide deletion or deletions, said nucleotide deletion or deletions being in one or more nucleotide positions chosen in the group comprising 1, 2, 14, 15, 16, 17, 18, 19, 20, 21 and 22, the numbering referring to the nucleotide numbering in SEQ ID NO: 66.

In certain additional embodiments, the aptamer used according to the invention can include a polynucleotide of SEQ ID NO: 66, or a polynucleotide that has a nucleotide sequence that differs from the SEQ ID NO: 66 at a rate of one of the following combinations of nucleotide deletions:
deletions of nucleotides at the positions 19, 20, and 21,
deletions of nucleotides at the positions 18, 19, 20 and 21,
deletions of nucleotides at the positions 15, 16, 19 and 20,
deletions of nucleotides at the positions 14, 15, 16, 20 and 21, and
deletions of nucleotides at the positions 14, 15, 16, 20, 21 and 22. the numbering referring to the nucleotide numbering in SEQ ID NO: 66.

Alternatively or in addition, the nucleotide deletions can be present at the positions 1 and 2, the numbering referring to the nucleotide numbering in SEQ ID NO: 66.

As shown in example 7, the applicant has identified variants of SEQ ID NO: 66 that can bind to the fibrinogen concurrently.

In certain additional or alternative embodiments, the aptamer used according to the invention is an aptamer that binds selectively to the fibrinogen and that comprises a polynucleotide chosen from the group comprised of SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, and SEQ ID NO: 93 or having a nucleotide sequence that differs at a rate of 1, 2, 3, 4 or 5 nucleotide modifications in relation to a sequence chosen in the group comprised of SEQ ID NO: 80-93.

Preferred variants of SEQ ID NO: 66 encompass aptamers of SEQ ID NO: 80-87.

The first subgroup of aptamers used according to the invention also encompasses aptamers directed against fibrinogen and which comprises having a sequence identity of at least 80%, 85%, 90%, 92%, 94%, 95%, 96%, 97% or 98% with the SEQ ID NO: 1.

In particular, the aptamer can be of SEQ ID NO: 1 or it can have a nucleotide sequence that differs from the SEQ ID NO: 1 at a rate of 1-20, in particular of 1, 2, 3, 4, 5, 6, 7, 8, 9 or 10 nucleotide modifications. Said nucleotide modification or modifications can be more preferably present at the position or positions described hereinabove for the SEQ ID NO: 66.

As explained in the section hereinbelow entitled “Method for obtaining aptamers used according to the invention”, certain aptamers used according to the invention have been identified from a mixture of candidates constituted of a multitude of single-stranded DNAs (ssDNA), wherein each ssDNA comprises a random central sequence from about 20 to 100 nucleotides flanked with specific sequences of from about 15 to 40 nucleotides that play the role of primers for an amplification via PCR.

In certain alternative or additional embodiments, the aptamer used according to the invention responds to the formula (I) wherein:

5′-[NUC1]m-[CENTRAL]-[NUC2]n-3′ (I)

wherein:
[CENTRAL] is a polynucleotide having a sequence identity of at least 70%, more preferably of at least 80%, for example of at least 85%, 90%, 93%, 95%, 96%, 97%, 98%, 99% or 100%, with the SEQ ID NO: 94,
n and m are integers chosen independently from 0 and 1,
[NUC1] is a polynucleotide comprising from 2 to 40 nucleotides, more preferably from 15 to 25 nucleotides, and
[NUC2] is a polynucleotide comprising from 2 to 40 nucleotides, more preferably from 15 to 25 nucleotides.

In a preferred embodiment, m=1 and [NUC1] comprises, or is constituted of a polynucleotide of SEQ ID NO: 75 or which differs from SEQ ID NO: 75 at a rate of 1, 2, 3 or 4 nucleotide modifications.

An example of an aptamer of formula (I) is the aptamer of SEQ ID NO: 66.

Consequently, the aptamer used according to the invention responds to the following formula:

5′-[NUC1]-[CENTRAL]-[NUC2]n-3′

wherein n is 0 or 1.

In certain other embodiments, [NUC2] can include, or is constituted of a polynucleotide of SEQ ID NO: 76 or of a polynucleotide that differs from SEQ ID NO: 76 at a rate of 1, 2, 3 or 4 nucleotide modifications.

In a specific aspect, the aptamer used according to the invention can be an aptamer of formula (I):

5′-[NUC1]m-[CENTRAL]-[NUC2]n-3′ (I)

wherein:
m is 1,
n is 0 or 1,
[NUC1] is a polynucleotide of SEQ ID NO: 75 or which differs from SEQ ID NO: 75 at a rate of 1, 2, 3 or 4 nucleotide modifications, more preferably nucleotide deletions, and
[NUC2] is a polynucleotide having a length from 2 to 40 nucleotides, and
[CENTRAL] is the polynucleotide of SEQ ID NO: 94, or has a sequence of nucleotides which differs from SEQ ID NO: 94 at a rate of 1, 2, 3, 4, 5 or 6 nucleotide modifications, more preferably nucleotide deletions.

In certain embodiments, [NUC2] is a polynucleotide of SEQ ID NO: 76, or has a sequence which differs from SEQ ID NO: 76 at a rate of 1, 2, 3 or 4 nucleotide modifications, said nucleotide modifications being more preferably chosen from nucleotide substitutions and nucleotide deletions.

In certain embodiments, the aptamers of said first subgroup can include a conformation group, as shown in FIG. 1A by the underlined nucleotides. In a more general aspect, the aptamers used according to the invention can have a sequence of nucleotides comprising nucleotide domains that can form a conformation group comprising a central loop that comprises from 15 to 19 nucleotides, more preferably 17 nucleotides bearing:
a first rod having a length from 4 to 6 nucleotides, more preferably of 5 nucleotides,
a second rod having from 2 to 4 nucleotides, more preferably 3 nucleotides connected to a loop comprising from 13 to 15, more preferably 14, nucleotides, and
possibly a third rod having from 2 to 8, more preferably 7 nucleotides connected to a loop comprising from 2 to 4, more preferably 3, nucleotides.

In certain preferred embodiments, the first rod is adjacent to the second rod and separated by 2 nucleotides coming from the third rod.

The aptamers belonging to the first subgroup of the invention can be able to bind to the fibrinogen at a slightly acidic pH defined hereinabove, more preferably at a pH of about 6.3. In particular, the aptamers of the first group can bind to the fibrinogen in a pH-dependent manner.

In certain embodiments, said aptamers have an increased affinity for the fibrinogen at pH 6.3 in relation to a slightly alkaline pH such as pH 7.4. In certain embodiments, said aptamer does not bind to the fibrinogen at a pH of 7.0 or more.

Said subgroup of aptamers can also be able to bind to the fibrinogen in the presence of Mg2+. In certain embodiments, said aptamers can have a binding affinity for the fibrinogen that depends on the pH and/or on the presence of Mg2+ in the medium. For example, the binding affinity of the aptamer for the fibrinogen can be increased in the presence of Mg2+ at a concentration in the range of mM, for example from 1 to 10 mM, in relation to the same medium devoid of Mg2+. As another example, the aptamer used according to the invention binds specifically to the fibrinogen at a pH of about 6.3 and does not bind to the fibrinogen at a pH higher than 7.0, such as pH 7.4.

These properties are for example shown here for the aptamer of SEQ ID NO: 66 in the section hereinbelow entitled “Examples”.

### —Second Subgroup of Aptamers Used According to the Invention

The second subgroup of aptamers encompasses, without being limited thereto, the aptamers of SEQ ID NO: 58-65 and the base sequence of SEQ ID NO: 67.

The aptamers of SEQ ID NO: 58-65 and of SEQ ID NO: 67 include two consensus groups in their sequences, namely GTTGGTAGGG (SEQ ID NO: 77) which is upstream of GGTGTAT (SEQ ID NO: 78), as shown in the alignment of sequences of FIG. 1C. Without intending to be bound by any theory, the applicant thinks that these consensus groups are located in a region of the aptamers that forms a loop shown in FIG. 1B for the aptamer of SEQ ID NO: 58. This conformational group can play a role in the binding of said aptamers to the fibrinogen. Consequently, this second subgroup of aptamers encompasses aptamers that specifically bind to the fibrinogen and which comprises the nucleotide groups of SEQ ID NO: 77 and of SEQ ID NO: 78, SEQ ID NO: 77 being upstream of SEQ ID NO: 78 in the base sequence of said aptamer.

Preferably, said aptamer comprises a nucleotide group of formula (III):

5′-[SEQ ID NO: 79]-[X1]-[SEQ ID NO: 77]-[X2]-[SEQ ID NO: 78]-3′

wherein:
[X2] and [X1] independently designate a nucleotide or an oligonucleotide of a length from 2 to 5 nucleotides, more preferably of a length of 2 or 3 nucleotides,
[SEQ ID NO: 77] is an oligonucleotide of SEQ ID NO: 77 (namely GTTGGTAGGG),
[SEQ ID NO: 78] is an oligonucleotide of SEQ ID NO: 78 (namely GGTGTAT) and
[SEQ ID NO: 79] is an oligonucleotide of SEQ ID NO: 79 (namely TGT).

For example, the aptamer used according to the invention can include a group of formula (III) wherein X1 designates a nucleotide, for example G or T, and X2 is an oligonucleotide of a length of 3 nucleotides. In certain embodiments, the aptamers can furthermore include a polynucleotide having a sequence identity of at least 70%, 75%, 80%, 85%, 90% or 95%, with a sequence chosen in the group constituted of SEQ ID NO: 67, SEQ ID NO: 68, SEQ ID NO: 69, SEQ ID NO: 70, SEQ ID NO: 71, SEQ ID NO: 72, SEQ ID NO: 73 SEQ ID NO: 74, and SEQ ID NO: 95.

Consequently, the second subgroup of aptamers encompasses aptamers that specifically bind to the fibrinogen and which comprises a polynucleotide:
having a sequence identity of at least 70%, 75%, 80%, 85%, 90% or 95%, with a sequence of nucleotides chosen from the group comprising SEQ ID NO: 67, SEQ ID NO: 68, SEQ ID NO: 69, SEQ ID NO: 70, SEQ ID NO: 71, SEQ ID NO: 72, SEQ ID NO: 73 SEQ ID NO: 74, and SEQ ID NO: 95, and
comprising a nucleotide group of formula (III):

5′-[SEQ ID NO: 79]-[X1]-[SEQ ID NO: 77]-[X2]-[SEQ ID NO: 78]-3′

wherein:
[X2] and [X1] independently designate a nucleotide or an oligonucleotide of a length from 2 to 5 nucleotides, more preferably of a length of 2 or 3 nucleotides,
[SEQ ID NO: 77] is an oligonucleotide of SEQ ID NO: 77 (namely GTTGGTAGGG),
[SEQ ID NO: 78] is an oligonucleotide of SEQ ID NO: 78 (namely GGTGTAT) and
[SEQ ID NO: 79] is an oligonucleotide of SEQ ID NO: 79 (namely TGT).

The aptamer used according to the invention has more preferably a length from 20 to 150 nucleotides, in particular a length from 25 to 100 nucleotides, such as a length of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99.

In an additional or alternative embodiment, the aptamer used according to the invention binds to the fibrinogen and comprises a polynucleotide having a sequence identity of at least 70%, 75%, 80%, 85%, 90% or 95% with the base sequence of SEQ ID NO: 67.

In certain embodiments, the aptamer used according to the invention binds specifically to the fibrinogen and comprises a polynucleotide SEQ ID NO: 67, or which differs from SEQ ID NO: 67 at a rate of 1, 2, 3, 4, 5 or 6 nucleotide modifications.
In a particular aspect, the aptamer used according to the invention binds specifically to the fibrinogen and responds to the formula (I) 5′-[NUC1]m-[CENTRAL]-[NUC2]n−3′ wherein n and m are integers chosen independently from 0 and 1,
[NUC1] is a polynucleotide comprising from 2 to 40 nucleotides, more preferably from 15 to 25 nucleotides,
[NUC2] is a polynucleotide comprising from 2 to 40 nucleotides, more preferably from 15 to 25 nucleotides and
[CENTRAL] is a polynucleotide having a sequence identity of at least 70%, 75%, 80%, 85%, 90% or 95% with a sequence of nucleotides chosen from the group comprising SEQ ID NO: 68, SEQ ID NO: 69 SEQ ID NO: 70, SEQ ID NO: 71, SEQ ID NO: 72, SEQ ID NO: 73, SEQ ID NO: 74, SEQ ID NO: 94 and SEQ ID NO: 95 and which comprises a nucleotide group of formula (III) defined hereinabove.

In certain embodiments, the aptamer used according to the invention is an aptamer of formula (I) comprising at least 1, 2, 3 or all of the following characteristics:
n=m=1,
[NUC1] comprises, or is constituted of a polynucleotide of SEQ ID NO: 75 or of a polynucleotide that differs from SEQ ID NO: 75 at a rate of 1, 2, 3 or 4 nucleotide modifications,
[NUC2] comprises, or is constituted of a polynucleotide of SEQ ID NO: 76 or of a polynucleotide that differs from SEQ ID NO: 76 at a rate of 1, 2, 3 or 4 nucleotide modifications,
[CENTRAL] is a polynucleotide having a sequence identity of at least 80%, more preferably of at least 85%, with SEQ ID NO: 95 and which comprises a nucleotide group of formula (III) defined hereinabove.

In another alternative or particular aspect, the aptamer used according to the invention binds specifically to the fibrinogen and comprises a polynucleotide having a sequence identity of at least 70%, 75%, 80%, 85%, 90% or 95% with a polynucleotide chosen from SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, and SEQ ID NO: 67.

In certain embodiments, the aptamers of said second subgroup can include a conformation group, as shown in FIG. 1B by the underlined nucleotides. In a more general aspect, the aptamers used according to the invention can have a sequence of nucleotides comprising nucleotide domains that can form a conformation group comprising a rod having a length from 2 to 5 nucleotides, for example 4 nucleotides, bound to a loop from 23 to 27 nucleotides. Preferably, the loop is devoid of any rod-loop group or additional rod group.

The aptamers belonging to said second subgroup can be able to bind to the fibrinogen at a slightly acidic pH, more preferably at a pH of about 6.3. In certain embodiments, said aptamers having an increasing binding with the fibrinogen at pH 6.3 in relation to a pH higher than 7.0, such as pH 7.4. Preferably, said aptamers do not bind to the fibrinogen at a pH higher than 7.0. More generally, the aptamers of the second subgroup can bind to the fibrinogen in a pH-dependent manner.

This subgroup of aptamers can also be able to bind to the fibrinogen in the absence of Mg2+. In certain embodiments, said aptamers can have a binding affinity for the fibrinogen that depends on the pH and/or on the presence of Mg2+ in the medium. For example, the binding affinity of the aptamer for the fibrinogen can be reduced in the presence of Mg2+, for example in a medium comprising Mg2+ in the range of mM, in relation to the same medium devoid of Mg2+.

### —Use of Aptamers According to the Invention as Affinity Ligands

Affinity ligands comprising an aptamer directed against the fibrinogen can be used according to the invention. Said affinity ligands can be immobilised on a solid support for the purification of the fibrinogen.

Typically, the affinity ligand used according to the invention comprises (i) an aptamer group, namely an aptamer directed against the fibrinogen such as defined hereinabove bound to at least one (ii) non-aptamer entity useful for the immobilisation on a suitable substrate. Preferably, the non-aptamer entity is more preferably bound to the end 5′- or 3′ of the aptamer. In a certain embodiment, the affinity ligand can include a means of immobilisation bound to the aptamer group directly or by a spacer group. Consequently, the affinity ligand can include, or be constituted of a compound of formula (IV):

[IMM]-([SPACER])p-[APTAMER]wherein
[APTAMER] designates an aptamer such as defined hereinabove,
[SPACER] is a spacer group,
[IMM] is a group for the immobilisation of the aptamer on a support, and
p is 0 or 1.

p equal to 0 means that the spacer is absent and that [IMM] is directly bound to [APTAMER], more preferably to the end 3′ or 5′ of the aptamer.

p equal to 1 means that the spacer is present and binds [IMM] and [APTAMER].

The spacer group is typically chosen so as to reduce the steric hindrance of the aptamer group and so as to improve its accessibility while still retaining the capacity of the aptamer to specifically bind to the fibrinogen. The spacer group can be of any type. The spacer can be a non-specific single-stranded nucleotide, namely which is not able to bind to a protein, including fibrinogen. Typically, the spacer can have a length from 2 to 20 nucleotides. Examples of suitable nucleic spacers are polyA and polyT. In certain other embodiments, the spacer can be a non-nucleic chemical entity. For example, the spacer can be chosen in the group constituted of a peptide, a polypeptide, an oligo- or a polysaccharide, a hydrocarbon chain possibly interrupted by one or several heteroatoms and possibly substituted with one or several substituents such as a hydroxyl, halogens or alkyl in C1-C3; polymers including sequenced homopolymers, copolymers and polymers, and combinations of the latter. For example, the spacer can be chosen from the group comprising polyethers such as polyethylene glycol (PEG) or polypropylene glycol, polyvinyl alcohol, polyacrylate, polymethacrylate, polysilicone, and a combination of the latter. For example, the spacer can include several hydrocarbon, oligomer or polymer chains bound by any suitable group, such as a heteroatom, more preferably —O— or —S—, —NHC(O)—, —OC(O)—, —NH—, —NH—CO—NH—, —O—CO—NH—, a phosphodiester or a phosphorothioate. These spacer chains can include from 2 to 200 carbon atoms, such as from 5 to 50 carbon atoms. Preferably, the spacer is chosen from non-specific oligonucleotides, hydrocarbon chains, polyethers, in particular polyethylene glycol and combinations of the latter.

For example, the spacer comprises at least one polyethylene glycol group comprising from 2 to 20 monomers. For example, the spacer can include from 1 to 4 triethylene glycol blocks bound together by suitable binding segments. For example, the spacer can be a derivative of hydrophilic ethylamine triethylene glycol in C12. Alternatively, the spacer can be a hydrocarbon chain in C2-C20, in particular an alkyl chain in C2-C20 such as a methylene chain in C12.

The spacer is more preferably bonded to the end 3′ or to the end 5 of the aptamer group. [IMM] designates any suitable group that makes it possible to immobilise the affinity ligand on a substrate, more preferably a solid support. [IMM] depends on the type of interactions sought to immobilise the affinity ligand on the substrate.

For example, the affinity ligand can be immobilised thanks to specific non-covalent interactions including hydrogen bonds, electrostatic forces or Van der Waals forces. For example, the immobilisation of the affinity ligand on the support can depend on ligand/anti-ligand pairs (for example, antibody/antigen such as biotin/anti-biotin antibodies and digoxygenin/anti-digoxygenin antibodies, or ligand/receptor) and binding protein labels. A multitude of protein labels are well known to those skilled in the art and encompasses, for example, biotin (for a binding with streptavidin or avidin derivatives), glutathion (for a binding with proteins or with other substances bound to glutathion-S-transferase), maltose (for a binding with proteins or with other substances bound to a binding protein to maltose), lectins (for a binding to sugar groups), a c-myc label, a hemaglutinin antigen label (HA), a thioredoxin label, a FLAG label, a polyArg label, a polyHis label, a Strep label, a chitin binding domain, a cellulose binding domain, and similar. In certain embodiments, [IMM] designates biotin. Consequently, the affinity ligand used according to the invention is suitable for an immobilisation on supports on which avidin or streptavidin is grafted.

Alternatively, the affinity ligand can be suitable for a covalent grafting on a solid support. [IMM] can therefore designate a chemical entity comprising a chemical reactive group. The chemical entity typically has a molecular weight lower than 1000 g·mol−1, preferably lower than 800 g·mol−1, such as lower than 700, 600, 500 or 400 g·mol−1. The reactive groups can be of any type and encompass a primary amine, a maleimide group, a sulfhydryl group, and similar.

For example, the chemical entity can be derived from a SIAB compound, a SMCC compound or derivatives of the latter. The use of sulfo-SIAB to immobilise oligonucleotides is described, for example, in Allerson et al., RNA, 2003, 9:364-374.

In certain embodiments, [IMM] comprises a primary amino group. For example, [IMM] can be —NH2 or aminoalkyl in C1-C30, more preferably aminoalkyl in C1-C6. An affinity ligand wherein [IMM] comprises a primary group that is suitable for an immobilisation on a support bearing activated carboxylic acid groups. The activated carboxylic acid groups encompass, without being limited thereto, an acid chloride, mixed ester and anhydrous groups. A preferred active carboxylic acid group is N-hydroxysuccinimide ester.

As mentioned hereinabove, [IMM]-([SPACER])p is more preferably bonded to the end 3′ or to the end 5′ of the aptamer. The end of the aptamer group which is not bound to [IMM]-([SPACER])p can include a chemically modified nucleotide such as 2′-o-methyl- or 2′-fluoropyrimidine, 2′-ribopurine, phosphoramidite, an inverted nucleotide or a chemical group such as PEG or cholesterol. These modifications can prevent the degradation, in particular the enzymatic degradation of the ligands. In other embodiments, said free end of the aptamer (namely which is not bound to [IMM] or to [SPACER]) can be linked to a means of detection such as described hereinabove.

An affinity support able to selectively bind to the fibrinogen can also be used according to the invention, it comprises on this support a plurality of affinity ligands such as described hereinabove.

The affinity ligands can be immobilised on the solid support by non-covalent interactions or by one or more covalent bindings.

In certain embodiments, the affinity ligands are covalently grafted on said support. Typically, the grafting is carried out by reaction of the chemical reactive group present in the group [IMM] of the ligand with a chemical reactive group present on the surface of the solid support.

Preferably, the chemical reactive group of the ligand is a primary amine group and the one present on the solid support is an activated carboxylic acid group such as a carboxylic group activated by NHS (namely N-hydroxysuccimidyl ester). In this case, the grafting reaction can be carried out at a pH lower than 6, for example at a pH from 3.5 to 4.5, as shown in the example 4 and described in WO2012090183, of which the description is incorporated here as a reference.

The solid support of the affinity support can be of any type and is chosen according to the use considered.

For example, the solid support can be chosen from a support made from plastic, metal and inorganic such as glass, nickel/nickel oxide, titanium, zirconium oxide, silicon, stressed silicon, polycrystalline silicon, silicon dioxide or a ceramic. Said support can be contained in a device such as a micro-electronic device, a microfluid device, a sensor, a biosensor or a chip, for example, suitable for use with SPR. Alternatively, the support can be in the form of beads, such as polymer, metal or magnetic beads. These supports can be suitable for detection and diagnostic needs.

In other embodiments, the solid support can be a polymer gel, a filter or a membrane. In particular, the solid support can be constituted of agar, crosslinked agar, cellulose or synthetic polymers such as polyacrylamide, polyethylene, polyamide, polysulfone, and the derivatives of the latter. These supports can be suitable for the purification of the fibrinogen. For example, the solid support can be a support for a chromatography, in particular can be a support for a liquid affinity chromatography. For example, the affinity support used according to the invention can be suitable for the carrying out of an affinity chromatography on an industrial scale. The affinity support used according to the invention can therefore be used as a stationary phase in a chromatography method, for example in a column chromatography method or in a discontinuous chromatography method.

Use of Aptamers and of Affinity Ligands According to the Invention in the Purification of the Fibrinogen

A method for capturing the fibrinogen used according to the invention comprises the following steps:
the supplying of a solid support on which an aptamer or an affinity ligand used according to the invention is immobilised,
the putting into contact of said solid support with a solution containing the fibrinogen, so that the fibrinogen is captured by the formation of a complex between the fibrinogen and said aptamer or said affinity ligand immobilised on the solid support.

In certain embodiments, the method can include one or several additional steps such as:
a step of releasing the fibrinogen from said complex,
a step of recovering the fibrinogen from said complex,
a step of detecting the formation of the complex between the fibrinogen and said aptamer or affinity ligand,
a step of quantifying the fibrinogen.

The detection of the complex and the quantification of the fibrinogen (or that of the complex) can be carried out by any method known to those skilled in the art. For example, the detection and the quantification can be carried out by SPR, as shown in the examples.

Alternatively, it is possible to use a test of the ELISA type wherein a marked anti-fibrinogen antibody is used for the detection or the quantification of the complex formed between the fibrinogen and the affinity ligands according to the invention. The anti-fibrinogen antibody can be marked with a fluorophore or coupled to an enzyme suitable for the detection, such as horseradish peroxidase.

As shown in detail in the examples 5 and 6 hereinbelow, the aptamers used according to the invention are particularly suitable for a use in the purification of the fibrinogen.

A method of purification of the fibrinogen used according to the invention using a starting composition comprises the following steps:
a. the putting into contact of said starting composition with an affinity support such as defined hereinabove, in conditions that are suitable for the formation of a complex between (i) the aptamers or the affinity ligands immobilised on said support and (ii) the fibrinogen,
b. the releasing of the fibrinogen from said complex, and
c. the recovering of the fibrinogen in a purified form.

A method of preparation used according to the invention for a purified fibrinogen composition using a starting composition containing the fibrinogen comprises the following steps:

a. the putting into contact of said starting composition with an affinity support such as defined hereinabove, in conditions that are suitable for the formation of a complex between (i) the aptamers or the affinity ligands immobilised on said support and (ii) the fibrinogen,
b. the releasing of the fibrinogen from said complex, and
c. the recovering of the purified fibrinogen composition.

As is meant here, the starting composition can be any composition that potentially comprises fibrinogen. The starting composition may contain contaminants from which the fibrinogen has to be separated.

The contaminants can be of any type and depend on the nature of the starting composition. The contaminants encompass proteins, salts, hormones, vitamins, nutrients, lipids, cell debris such as fragments of cell membranes, and similar. In certain embodiments, the contaminants can include blood proteins such as coagulation factors, fibronectin, albumin, immunoglobulin, a plasminogen, alpha-2-macroglobulin, and similar.

In certain other embodiments, the contaminant can include non-human proteins, in particular non-human proteins expressed endogenously by a recombinant host such as a recombinant cell, a bacterium or a yeast, or a transgenic animal.

Typically, the starting composition can be, or can derive, from a cell culture, from a fermentation broth, from a cell lysate, from a tissue, from an organ or from a body fluid. As is meant here, a “starting composition” is derived from an entity of interest, such as milk, blood or a cell culture, which means that the starting composition is obtained from said entity by submitting said entity to one or several treatment steps. For example, the entity of interest can be submitted to one or several treatments such as a cell lysate, a step of precipitation such as the precipitation of a salt, a cryo-precipitation or a flocculation, a step of filtration such as a forced filtration or an ultrafiltration, a centrifugation, a clarification, a chromatography, a step of extraction such as an liquid-liquid or solid-liquid extraction, a viral inactivation, a pasteurisation, steps of freezing/defrosting, and similar. For example, a starting composition derived from blood encompasses, without being limited thereto, plasma, a plasma fraction and a blood cryoprecipitate.

In certain embodiments, the starting solution is derived from blood, more preferably human blood. The starting composition can be chosen from plasma, a plasma fraction, for example the fraction I obtained by an ethanol fractionation Cohn method, and a blood cryoprecipitate. In certain embodiments, the starting composition is an immunoglobulin depleted plasma fraction and/or an albumin depleted plasma fraction and/or a blood or plasma fraction depleted in vitamin K dependent coagulation protein.

In certain other embodiments, the starting composition is obtained from a recombinant host. Preferably, the recombinant host is a transgenic animal, such as a non-human transgenic mammal. The non-human transgenic mammal can be any animal that has been genetically modified to express human fibrinogen. Preferably, the human fibrinogen is expressed in a body fluid of said transgenic animal.

The starting solution can therefore be, or can derive, from a body fluid of a transgenic animal. The body fluids encompass, without being limited thereto, blood, breast milk and urine.

In a particular embodiment, the starting composition is, or derives, from the milk coming from a non-human transgenic mammal. The production methods of a transgenic animal able to secrete a protein of interest in the milk are well-known in the technique. Typically, these methods encompass the introduction of a genetic assembly product comprising an encoding gene for the protein of interest optionally bonded to a promoter from a protein which naturally secreted in the milk (such as a casein promoter or a WHAP promoter) in an embryo of a non-human mammal. The embryo is then transferred to the uterus of a female belonging to the same animal species and which was prepared from a hormonal standpoint for a pregnancy.

In certain preferred embodiments, the starting composition can be chosen from human blood, transgenic milk and the derivatives of the latter.

The affinity support used in the methods of the invention can be any affinity support described hereinabove. Preferably, the affinity support is an affinity support for the carrying out of an affinity chromatography. Indeed, the methods of purification of the fibrinogen or for preparing a purified composition of fibrinogen are more preferably based on chromatography technologies, for example discontinuous modes or on a column, wherein the affinity support plays the role of a stationary phase. In a step a), a suitable volume of the starting composition containing the fibrinogen is placed in contact with an affinity support in suitable conditions to favour the specific interactions of anti-fibrinogen aptamer groups present on the surface of the affinity support with the fibrinogen, in such a way that a complex is formed between the molecules of fibrinogen and said aptamer groups. In the step a), the fibrinogen is therefore retained on the affinity support. The bond between the aptamer groups and the molecules of fibrinogen can be reinforced by carrying out the step a) at a slightly acidic pH. In certain embodiments, the step a) is carried out at a pH lower than 7.0, preferably lower than 6.9, 6.8 or 6.7. In particular, the step a) can be carried out at a pH from 6.0 to 6.8, more preferably at a pH from 6.0 to 6.5, such as 6.0, 6.1, 6.2, 6.3, 6.4 or 6.5. For example, the step a) can be carried out at a pH from 6.1 to 6.5, such as a pH of 6.3. In a more general aspect, the pH condition of the step a) can be chosen in such a way as to favour the binding of the fibrinogen on the affinity support while still minimising the binding of the other molecules on the affinity support. Typically, the step a) is carried out in the presence of a buffer solution (called in what follows “binding buffer”). The binding buffer can be mixed with the starting composition before the step a) or it can be added during the step a). The binding buffer is typically an aqueous solution containing a buffer agent. The buffer agent can be chosen in such a way as to be compatible with the fibrinogen and the affinity support and in such a way as to give the desired pH for the step a). For example, in order to obtain a pH of about 6.3, the buffer agent can be chosen, without being limited thereto, from 3-(N-morpholino)propanesulfonic acid (MOPS), 2-(N-morpholino)ethanesulfonic acid (MES), HEPES, Bis-TRIS, citrate and acetate. The buffer agent can be present at a concentration of about 5 mM to 1 mM, for example from 10 mM to 500 mM, for example from 10 mM to 200 mM, such as about 50 mM.

Without intending to be bound by any theory, the presence of salts can favour the formation of the complex between the fibrinogen and the aptamer groups of the solid support and/or prevent the binding of the other molecules present in the starting composition. Typically, the step a) can be carried out in the presence of sodium chloride, for example at a concentration ranging from 10 mM to 500 mM, more preferably from 50 mM to 350 mM, or from 100 mM to 200 mM, such as about 150 mM.

The presence of divalent cations can modulate the binding of the fibrinogen to the aptamer groups. In certain embodiments, the step a) is carried out in the presence of divalent cations, such as Mg2+, at a concentration of at least 1 mM, for example at a concentration of about 1 mM to 50 mM, for example from 1 mM to 20 mM, such as a concentration of about 5 mM.

In certain other embodiments, the step a) is carried out in the absence of Mg2+; and more generally, in the absence of divalent cations.

Consequently, the binding buffer used in the step a) can include NaCl at a concentration from 100 mM to 500 mM and a magnesium salt such as magnesium chloride (MgCl2) at a concentration of about 1 mM to 50 mM, and it can have a pH of 5.0 or of 6.9. This buffer can be suitable when the aptamer groups present on the solid support are chosen in the first subgroup of the invention defined hereinabove.

A binding buffer suitable for carrying out the step a), in particular when the aptamer group belongs to the first subgroup defined hereinabove, can be a buffer comprising 50 mM of MOPS, 5 mM of MgCl2 and 150 mM of NaCl, at pH 6.3.

When the aptamer groups present on the affinity support are chosen in the second subgroup of aptamers defined hereinabove, the binding buffer can be devoid of Mg2+, and more generally of divalent cations. A binding buffer suitable for carrying out the step a) can therefore be a buffer comprising 50 mM of MOPS and 150 mM of NaCl, at pH 6.3.

At the end of the step a), and before the step b), the affinity support can be washed with a suitable wash buffer in such a way as to eliminate substances that are not specifically bound, but adsorbed on the support. It goes without saying that wash buffer does not significantly alter the complex between the fibrinogen and the aptamer group while still favouring a desorption of the substances that are not specifically bound to the affinity support.

Thus, in certain embodiments, the method of the invention comprises a step of washing the affinity support at the end of the step a) and before the step b). Any conventional wash buffer, well known to those skilled in the art, can be used. In certain embodiments, the wash buffer has the same composition as that of the binding buffer used in the step a). In other embodiments, the wash buffer can include the same components, but at different concentrations, in relation to the binding buffer used in the step a). In additional or alternative embodiments, the pH of the wash buffer is the same as that of the binding buffer.

The wash buffer can have a pH lower than 7, for example from pH 5.0 to 6.9, more preferably from 6.1 to 6.5, such as pH 6.3. The wash buffer can furthermore include NaCl. Typically, the ionic force of the wash buffer can be higher than that of the binding buffer. Indeed, the applicant has shown that, for certain aptamers of the invention, a high ionic force may not significantly alter the binding of the fibrinogen to the aptamer groups. In other terms, the complex between the fibrinogen and certain aptamers used according to the invention can be stable, even in the presence of a high ionic force. Thus, in certain embodiments, the wash solution has an ionic force higher than that of the binding buffer used in the step a). In alternative or additional embodiments, the wash buffer can include a concentration of NaCl of at least 100 mM and up to 10 M. For example, the concentration of the NaCl can be about 100 mM to 5 mM, more preferably from 150 mM to 2 mM. Possibly, the wash buffer further comprises divalent cations, in particular Mg2+, at a concentration of about 0.1 mM to 20 mM, more preferably from 1 mM to 10 mM, such as a concentration of about 5 mM. In certain embodiments, the wash buffer is devoid of Mg2+ and more generally of divalent cations.

In certain other additional embodiments, the wash buffer can include at least one additional component, more preferably chosen from alkyldiols, in particular from ethylene glycol or propylene glycol. Indeed, for certain aptamers used according to the invention, the presence of alkyldiols such as ethylene glycol in the wash solution does not alter the complex between the fibrinogen and the aptamer. The wash buffer can therefore include an alkyldiol such as ethylene glycol or propylene glycol in a quantity from 1% to 70% by weight, more preferably from 10% to 60% by weight, such as 50% by weight.

Solely for the purposes of illustration, the wash buffer comprises MOPS 50 mM, NaCl 2M at pH 6.3 and possibly 50% by weight of glycol. This wash buffer can be suitable for the carrying out of the washing of an affinity support bearing aptamer groups belonging to the second subgroup of aptamers described hereinabove. Another example of wash buffer is a solution comprising MOPS 50 mM, NaCl 0.5M and MgCl2 5 mM at pH 6.3.

The step b) has for purpose to release the fibrinogen from the complex formed in the step a). This releasing can be obtained by destabilisation of the complex between the fibrinogen and the aptamer groups, namely using conditions that reduce the affinity of the aptamers for the fibrinogen. It should be noted that the complex between the aptamer group and the fibrinogen can be destabilised in moderate conditions that are not able to alter the fibrinogen.

As explained hereinabove, the capacity of the aptamers used according to the invention to bind to the fibrinogen can depend on the pH of the medium. An increase in the pH beyond 7.0 can make it possible to favour the release of the fibrinogen. Thus, in certain embodiments, the step b) is carried out by increasing the Ph beyond 7.0. Preferably, the pH of the step b) is from 7.0 to 8.0, for example from 7.2 to 7.8, such as a pH of 7.4. In other terms, an elution buffer at a pH higher than 7.0 can be used to favour the release of the fibrinogen. Solely for the purposes of illustration, a suitable elution buffer can be a solution buffered with MOPS 50 mM at pH 7.4 and comprising NaCl 150 mM.

As explained hereinabove, the capacity of the aptamer to bind to the fibrinogen can also vary according to the presence of divalent cations, such as Mg2+. For example, the binding of the aptamer group to the fibrinogen can be favoured by the presence of Mg2+. Thus, the release of the protein from the complex in the step b) can be favoured by using an elution buffer devoid of divalent cations and/or comprising an agent that chelates the divalent cations, such as EDTA or EGTA. For example, that agent that chelates the divalent cations can be present at a concentration of at least 1 mM and of at most 500 mM in the elution buffer used in the step b). The utilisation of a chelating that chelates the divalent cations can be suitable for an affinity support bearing aptamers belonging to the first subgroup described hereinabove.

In other embodiments, the binding of the aptamer group to the fibrinogen can decrease in the presence of divalent cations such as Mg2+. Thus, in this embodiment, the elution buffer can include divalent cations, in particular Mg2+, at a concentration of about 0.1 mM to 20 mM, more preferably from 1 mM to 10 mM, such as a concentration of about 5 mM. This elution can be suitable for the release of the protein from the complex formed with an aptamer group belonging to the second subgroup defined hereinabove.

Another example of an elution buffer that can be used in the step b) is a solution of MOPS 50 mM at pH 7.4 comprising MgCl2 2 M.

At the end of the step c), the purified fibrinogen is typically obtained in the form of a purified liquid composition. This purified liquid composition can be subjected to one or more additional steps. Said liquid composition can be concentrated and/or subjected to an inactivation or a viral elimination, for example via a sterile filtration or via a detergent, a diafiltration, a step of formulation with one or several pharmaceutically acceptable excipients, a lyophilisation, a packaging, more preferably in sterile conditions, and combinations of the latter.

In a more general aspect, the method of purification of the fibrinogen or the method of preparation of a purified fibrinogen composition can include one or several additional steps including, without being limited thereto, one or several steps of chromatography such as an exclusion chromatography, an ion exchange chromatography, a multimodal chromatography, a reversed-phase chromatography, a hydroxylapatite chromatography or an affinity chromatography, a step of precipitation, one or several steps of filtration such as a forced filtration, an ultrafiltration, a tangential ultrafiltration, a nanofiltration, and a reverse osmosis, a step of clarification, a step on inactivation or of viral elimination, a sterilisation, a formulation, a lyophilisation, a packaging and combinations of the latter.

In certain additional embodiments, the method of purification of the fibrinogen or the method of preparation of a purified fibrinogen composition comprises one of the following combinations of characteristics:
combination 1: (i) the aptamer group is chosen from the aptamers of the first subgroup defined hereinabove, (ii) the step (a) is carried out at pH 5.8 to 6.5, more preferably 6.3, in the presence of Mg2+ and (iii) the step (b) is carried out at pH 7.0 to 8.0, more preferably 7.4, possibly in the presence of an agent that chelates the divalent cations, and
combination 2: (i) the aptamer group is chosen from the aptamers of the second subgroup defined hereinabove, (ii) the step (a) is carried out at pH 5.8 to 6.5, more preferably 6.3, in the absence of Mg2+ and (iii) the step (b) is carried out at pH 7.0 to 8.0, more preferably 7.4, in the presence of Mg2+.

In an additional aspect, the aptamers and the affinity ligands of the invention can be used in a method of fractionation of the blood plasma. The method of fractionation of the blood plasma can include several successive steps of affinity chromatography, each step of affinity chromatography making it possible to recover a plasma protein of interest such as fibrinogen, an immunoglobulin, an albumin and other coagulation factors, such as vitamin K-dependent coagulation factors. The affinity ligands used in each step can be of any type, in particular aptamers. In this respect, the applicant has surprisingly shown that plasma proteins such as fibrinogen, albumin and immunoglobulin, can be recovered and purified using blood plasma by carrying out successive steps of affinity chromatography based on aptamers. Note that the method of fractionation of the blood plasma comprising successive steps of affinity chromatography based on aptamers makes it possible to obtain a concentrate of fibrinogen and a concentrate of immunoglobulin with a protein purity of at least 96%, and even of at least 99%, with outputs of about 9-12 g per litre of plasma for immunoglobulins and of 2-4 g per litre of plasma for fibrinogen. The applicant has furthermore shown that these good outputs and these good rates of purity can be achieved using raw blood plasma. In other terms, the steps of affinity chromatography based on aptamers can be carried out on a raw blood plasma without any pre-treatment such as an ethanol fractionation (Cohn method), a cryo-precipitation, a caprylate fractionation or a precipitation with PEG. Note that this method of fractionation makes it possible to avoid temporary intermediate cold storage.

Another object of the invention is therefore a method of fractionation of blood plasma comprising:

(a) a step of affinity chromatography in order to recover the fibrinogen wherein the affinity ligand is an aptamer that binds specifically to the fibrinogen, and
(b) a step of affinity chromatography in order to recover the immunoglobulins (Ig) wherein the affinity ligand is an aptamer that binds specifically to the immunoglobulins, wherein the steps of affinity chromatography (a) and (b) can be carried out in any order.

Preferably, immunoglobulins of isotope G are recovered in the step (b).

The step of affinity chromatography in order to recover the fibrinogen can be carried out before the affinity chromatography in order to recover the Ig and inversely. Consequently, in certain embodiments, the method of fractionation of blood plasma comprises the steps of:
subjecting blood plasma or a derivative of the latter to a step of affinity chromatography, wherein the affinity ligand is an aptamer that binds specifically to the fibrinogen, and
subjecting the non-retained fraction, which is substantially devoid of fibrinogen, to a step of affinity chromatography, wherein the affinity ligand is an aptamer that binds specifically to immunoglobulin.

It goes without saying that the steps hereinabove can include the recovery of the fibrinogen and Ig retained on the affinity support, respectively.

In certain other embodiments, the method of fractionation of blood plasma comprises the steps of:
subjecting blood plasma or a derivative of the latter to a step of affinity chromatography, wherein the affinity ligand is an aptamer that binds specifically to an Ig, and
subjecting the non-retained fraction, which is substantially devoid of Ig, to a step of affinity chromatography, wherein the affinity ligand is an aptamer that binds specifically to fibrinogen.

It goes without saying that the steps hereinabove can include the recovery of Ig and of fibrinogen retained on the affinity support, respectively.

In the method of the invention, the starting composition can be a blood plasma or derivatives of the latter. The derivatives of blood plasma encompass, without being limited thereto, a clarified blood plasma, a lipid-depleted blood plasma, a cryoprecipitate of blood plasma, a supernatant of a cryoprecipitate of blood plasma, a plasma fraction, and similar. In certain embodiments, the starting composition is a raw blood plasma.

Preferably, immunoglobulins of the isotope G are recovered. The immunoglobulins of the isotope G encompass IgG1, IgG2, IgG3 and IgG4. In certain embodiments, the aptamer directed against the immunoglobulin is able to specifically bind to the IgG, independently of the subcategories of IgG. In certain embodiments, several types of anti-IgG aptamers are used in order to recover all of the subcategories of IgG. Preferably, the fraction of IgG recovered in the method of fractionation of the invention has a distribution of the subcategories close to that of the starting blood plasma, i.e. it comprises from 50% to 70% of IgG1, from 25% to 35% of IgG2, from 2% to 8% of IgG3 and from 1% to 8% of IgG4.

In certain embodiments, the method of fractionation of blood plasma of the invention comprises one or several additional steps, in particular (c) a step of purification of the albumin.

The purified albumin can be recovered by any conventional method such as a chromatography including an affinity chromatography, an ion exchange chromatography and a precipitation with ethanol followed by a filtration.

For example, the step (c) can be a step of affinity chromatography wherein the affinity ligand is an aptamer qui binds specifically to albumin.

When the step (c) is present, the steps (a), (b) and (c) can be carried out in any order. In certain embodiments, the step (c) is carried out on the non-retained fraction obtained from the step (a) or of the step (b).

Any type of chromatography technology can be used pour to carry out the steps (a), (b) and (c) in the method of the invention, such as a discontinuous chromatography, a simulated moving bed chromatography (SMB). The preferred chromatography technologies are those that comprise the use of several columns such as simulated moving bed chromatography SMB.

The method of fractionation of blood plasma can include one or several additional steps including, without being limited thereto, a step of chromatography in order to remove the anti-A and/or anti-B antibodies, an ultrafiltration, a tangential ultrafiltration, a nanofiltration, a reverse osmosis, a clarification, a step of viral inactivation, a step of viral elimination, a sterilisation, polishing steps such as a formulation, or a lyophilisation or combinations of the latter. The method of the invention can also include one or several additional steps with the purpose of preventing and/or eliminating the fouling of the chromatography columns such as a sanitary treatment with an alkaline solution, for example with a solution of sodium hydroxide.

The invention also relates to a purified fibrinogen composition that can be obtained or obtained by a method of preparation of a purified fibrinogen composition according to the invention or by the method of fractionation of blood plasma according to the invention.

Another object of the invention is a purified fibrinogen composition that comprises at least 90% by weight, more preferably at least 91%, 92%, 93%, 94%, 95%, 96%, 97%, 98%, 99%, 99.1%, 99.2%, 99.3%, 99.4%, 99.5%, 99.5%, 99.6%, 99.7%, 99.8% or 99.9% by weight, in relation to the total weight of the proteins present in said composition. In certain embodiments, the composition of purified fibrinogen comprises human plasma fibrinogen, for example the fibrinogen obtained from human plasma. In this embodiment, said composition comprises at most 10%, more preferably at most 9%, 8%, 7%, 6%, 5%, 4%, 3%, 2%, 1%, 0.9%, 0.8%, 0.7%, 0.6%, 0.5%, 0.4%, 0.3%, 0.2% or 0.1% by weight of other plasma proteins, in particular from other human coagulation factors. In certain embodiments, the composition is substantially devoid of human coagulation factors other than human fibrinogen. In certain additional or alternative embodiments, said composition is devoid of factor XIII.

In certain embodiments, the composition of purified fibrinogen comprises human recombinant fibrinogen, for example the human fibrinogen produced by a recombinant host such as a recombinant cell or a transgenic animal. In this embodiment, said composition comprised at most 10%, more preferably at most 9%, 8%, 7%, 6%, 5%, 4%, 3%, 2%, 1%, 0.9%, 0.8%, 0.7%, 0.6%, 0.5%, 0.4%, 0.3%, 0.2% or 0.1% by weight of other proteins, in particular other non-human proteins coming from the recombinant host. In certain embodiments, the composition is substantially devoid of non-human proteins. In certain additional or alternative embodiments, said composition is devoid of any non-human equivalent of fibrinogen that can be found in the recombinant host.

The invention also relates to a pharmaceutical composition comprising a purified composition of human fibrinogen such as recombinant human fibrinogen or human plasma fibrinogen such as defined hereinabove, in combination with one or several pharmaceutically acceptable excipients. Said pharmaceutical composition, as well as the purified liquid composition of fibrinogen according to the invention, can be used in the treatment of coagulation disorders, in particular in the treatments of a fibrinogen acquired or congenital deficiency (hypo-, dys- or a-fibrinogemia). The composition of the invention can be used in the management of acute post-traumatic or post-surgical bleeding or in the management of a fibrinogen deficiency resulting from an acute renal failure.

Method for Obtaining Aptamers Used According to the Invention

The applicant carried out several SELEX strategies described in prior art in order to identify aptamers directed against the human fibrinogen. None of these strategies was successful, and a standard SELEX resulted in the identification of aptamers directed against a contaminant, which explains why lower than 1% of the purified composition of fibrinogen is used to carry it out.

In this context, the applicant conducted in-depth research in order to develop a new method of obtaining aptamers directed against “SELEX-resistant” proteins such as fibrinogen.

The applicant designed a new SELEX method that makes it possible to obtain aptamers that have a strong binding affinity for “SELEX-resistant” proteins, and that can be used as affinity ligands in a method of purification. This new SELEX method is characterised by a step of selection which is carried out in pH conditions that are suitable for the creation of “positive patches” on the surface of the target protein. In other terms, the method designed by the applicant is based on the reinforcing of the local interactions between the potential aptamers and the target protein by favouring positive charges over a superficial domain of the protein. This method can be carried out for proteins that have one or several superficial histidines, such as fibrinogen. The pH of the step of selection (namely the step wherein the target protein is put into contact with the candidate mixture of nucleic acids) must be chosen in such a way as to favour the protonation of at least one superficial histidine of the target protein. In the case of fibrinogen, the applicant has sown that suitable pH for the step of selection is a slightly acidic pH.

Consequently, of method of obtaining an aptamer used according to the invention that binds specifically to the fibrinogen, comprises the following steps:

a) the putting into contact of the fibrinogen with a candidate mixture of nucleic acids at a pH lower than 7.0, more preferably from 5.8 to 6.8,
b) the recovering of the nucleic acids that bind to the fibrinogen, while still eliminating the non-bound nucleic acids,
c) the amplifying of the nucleic acids obtained in the step (b) in order to produce a candidate mixture of nucleic acids that has an increased affinity for fibrinogen, and
d) the repeating of steps (a), (b), (c) until the obtaining of one or several aptamers directed against fibrinogen.

In the step (a), the candidate mixture of nucleic acids is generally a mixture of chemically-synthesised statistic nucleic acids. The candidate mixture can include from 108 to 1018, typically about 1015 nucleic acids. The candidate mixture can be a mixture of nucleic acids of DNA or a mixture of nucleic acids of RNA. In certain embodiments, the candidate mixture is constituted of a multitude of single-stranded DNAs (ssDNA), wherein each ssDNA comprises a central statistical sequence of about 20 to 100 nucleotides flanked with specific sequences of from about 15 to 40 nucleotides that play the role of primers for an amplification via PCR. In certain other embodiments, the candidate mixture is comprised of a multitude of nucleic acids of RNA, wherein each RNA comprises a central statistical sequence of about 20 to 100 nucleotides flanked with primer sequences of about 15 to 40 nucleotides for an amplification by RT-PCR. In certain embodiments, the candidate mixture of nucleic acids is constituted of non-modified nucleic acids, which means that the nucleic acids include only nucleotides that exist naturally. In certain other embodiments, the candidate mixture can include chemically-modified nucleic acids. In other terms, the nucleic acids can include one or several chemically modified nucleotides. In preferred embodiments, the candidate mixture is constituted of single-stranded DNAs.

The step a) is carried out in conditions that are favourable for the binding of the fibrinogen to nucleic acids that have an affinity for said fibrinogen. Preferably, the pH of the step a) is from 6.0 to 6.6, such as 6.1, 6.2, 6.3, 6.4 and 6.5. A suitable pH for the step a) is, for example, 6.3±0.1. This pH makes it possible to protonate at least one superficial histidine of the fibrinogen. The step (a) can be carried out in a buffered aqueous solution. The buffer agent can be chosen from any buffer agent that makes it possible to obtain the desired pH and that is compatible with the target proteins and the mixture of nucleic acids. The buffer agent can be chosen, without being limited thereto, from 3-(N-morpholino)propanesulfonic acid (MOPS), 2-(N-morpholino)ethanesulfonic acid (MES), HEPES, Bis-Tris, citrate and acetate. The buffer agent can be present at a concentration of about 5 mM to 1 M, for example from 10 mM to 500 mM, for example from 10 mM to 200 mM, such as about 50 mM.

In a particular embodiment of the invention, the liquid composition, stable and comprising fibrinogen is obtained according to the method comprising the following steps:
obtaining of a blood plasma cryosupernatant fraction,
precipitation of the cryosupernatant by 8% ethanol so as to obtain a fibrinogen-enriched fraction,
purification of the fibrinogen-enriched fraction of blood plasma after re-suspending by separation by affinity gel chromatography using preferably affinity ligands chosen from antibodies, fragments of antibodies, derivatives of antibodies or chemical ligands such as peptides, peptide mimetics, peptoids, nanofitins or oligonucleotide ligands such as aptamers,
recovery of the purified absorbed fraction comprising fibrinogen,
possibly, adding of pharmaceutically acceptable excipients, preferably arginine and/or citrate.

In a particular embodiment, the method further comprises a step of storage for at least 3 months at 4° C.

In a particular embodiment of the invention, the affinity chromatography used is an affinity matrix with ligands type moiety derived from llama antibody such as the Fibrinogen CaptureSelect matrix (Life Technologies).

Particularly advantageously, the composition according to the invention is devoid of proteases and/or activators of fibrinolysis.

It is understood that “fibrinogen composition devoid of proteases and/or of activators of fibrinolysis” means that the fibrinogen composition has been subjected to one or several steps that allow for the elimination of the proteases such as thrombin, prothrombin, plasmin, plasminogen in such a way that the residual quantity of proteases and/or of activators of fibrinolysis is:
very highly reduced in comparison with the pre-purified Fibrinogen solution before the step of chromatography, and/or
zero, and/or
lower than the detection thresholds of the methods that are commonly used by those skilled in the art.

Advantageously, the rate of residual prothrombin is lower than 5 μUI/mg of fibrinogen, the rate of plasminogen is lower than 15 ng/mg of fibrinogen.

In a particular embodiment of the invention, the composition according to the invention is therefore devoid of proteases such as thrombin and/or plasmin or their corresponding prothrombin pro-enzymes (factor II of coagulation) and/or plasminogen, which are zymogens that can potentially be activated.

In a particular embodiment of the invention, the fibrinogen composition according to the invention is devoid of inhibitors of proteases and/or anti-fibrinolytics.

The term “inhibitors of proteases and/or anti-fibrinolytics”, means any molecule with an antiproteasic activity, in particular any molecule with an inhibiting of serine protease and/or anti-fibrinolytic activity, in particular any molecule with an inhibiting activity of thrombin and/or anti-plasmin, in particular hirudin, benzamidine, aprotinin, phenylmethylsulfonyl fluoride (PMSF), pepstatin, leupeptin, antithrombin III associated or not with heparin, alpha 2 macroglobulin, alpha 1 antitrypsin, hexanoic or epsilon aminocaproic acid, tranexamic acid, alpha 2 antiplasmin.

In a particular embodiment of the invention, the fibrinogen composition according to the invention is devoid of hirudin and/or benzamidine and/or aprotinin and/or PMSF and/or pepstatin and/or leupeptin and/or antithrombin III associated or not with heparin and/or alpha 2 macroglobulin and/or alpha 1 antitrypsin and/or hexanoic acid and/or epsilon aminocaproic acid and/or tranexamic acid and/or alpha 2 antiplasmin.

In a particular embodiment of the invention, the fibrinogen composition according to the invention is devoid of metal ions.

In a particular embodiment of the invention, the composition according to the invention is advantageously devoid of calcium.

In a particular embodiment of the invention, the fibrinogen composition according to the invention is devoid of isoleucine, glycine and/or of NaCl.

In a particular embodiment of the invention, the fibrinogen composition according to the invention is devoid of albumin.

Advantageously, the composition according to the invention has a purity higher than or equal to 70%, preferably higher than or equal to 75%, 80%, 85%, 90%, 95%, 96%, 97%, 98%, 99%.

In a particular embodiment of the invention, the composition according to the invention does not contain other co-purified proteins, advantageously no FXIII and/or fibronectin.

In another particular embodiment of the invention, the fibrinogen composition according to the invention can also include one or several accompanying proteins, possibly co-purified. In a particular embodiment of the invention, the composition according to the invention advantageously comprises FXIII.

The composition according to the invention is subjected, directly after purification, to the step of producing the pharmaceutical form in liquid form: formulation, sterilising filtration and distribution into containers (bottle or other device for storage/administration).

Particularly advantageously, the composition according to the invention is not subjected to any step of freeze-drying, desiccation, dehydration or drying.

Particularly advantageously, the composition according to the invention is therefore in liquid form without having subjected a step of reconstitution of a lyophilisate.

Particularly advantageously, the composition according to the invention is in liquid form, and therefore comprises, in addition to the possible pharmaceutically acceptable excipients, water.

In a particular embodiment of the invention, the composition in liquid form comprises citrate and/or arginine, more preferably lower than 300 mM of arginine.

Advantageously, the composition according to the invention is particularly suited for intravenous administration.

Another aspect of the invention relates to the composition according to the invention for the use thereof in therapy. All of the technical characteristics mentioned hereinabove apply here.

The following example shows an embodiment of this invention without however limiting the scope of it.

### Example 1: Composition of Stable Liquid Fibrinogen

### Material & Method

### Plasma Fraction

The starting material is a pool of human plasma that was first subjected to a step of cryo-precipitation then subjected to a step of precipitation with 8% ethanol.

The pre-purified plasma fibrinogen solution thus obtained is diluted by 2 volumes of an equalising buffer of the gel before injection onto the chromatography column that is pre-balanced in pH and in conductivity.

Characteristics of the plasma fraction: antigen fibrinogen adjusted via dilution to 4.7-4.8 g/L.

### Buffer Solutions

The composition of the buffer solutions used during the various steps of the chromatography method is summarised in table 1 hereinbelow.

TABLEAU 1
Buffer solutions for the affinity chromatography.

| Phases | Composition | Target values |
|---|---|---|
| Balancing of the column | Tri-sodium citrate 10 mM, | pH adjusted |
| and return to the base line | Arginine HCl 0.1M | to 7.4 |
| Pre-elution | Tri-sodium citrate 10 mM, | pH adjusted |
| | Sodium Chloride 2.0M | to 7.4 |
| | Arginine HCl 0.1M | |
| Elution | Tri-sodium citrate 10 mM, | pH adjusted |
| | Propylene glycol 50% v/v | to 7.4 |
| | Arginine HCl 1.0M | |

### Affinity Chromatography Gels

For the chromatography, an affinity gel CaptureSelect Fibrinogen (Life Technologies ref 191291050, lots 171013-01 and 171013-05) is used, having an affinity ligand constituted of a fragment of anti-fibrinogen llama antibody.

### Columns

A column 50 mm in diameter (Reference XK 50/30 GE Healthcare), gel-packed volume of 67 ml; for a column height of 3.4 cm was used

### Purification by Affinity

The solution of fibrinogen adjusted to about 5 g/L is injected without any other adjustment on the balanced affinity column CaptureSelect Fibrinogen. A load of about 10 g/L was applied.

The chromatography eluate was then ultrafiltered and pre-formulated on the cut-off threshold membrane 100 kDa (reference Pall Omega OS100C10)

At the end of the method, the product obtained has a concentration in coagulable fibrinogen of 15.2 g/L and in antigen fibrinogen of 15.2 mg/ml.

### Example 2: Stability of a Composition of Liquid Fibrinogen

### Composition of Fibrinogen

The fibrinogen composition obtained according to the method illustrated in the example 1 is formulated at least with arginine 100 mM and citrate 8.5 mM at pH 7.0.

### Stability of the Fibrinogen Composition

The composition is placed into stability, under air (in the open air), without stirring, in a cold room at +5° C.

Samples are taken before stability testing (TO) and at different times according to the beginning of stability testing: two months (T2M) and 3 months (T3M).

### Results of Stability Testing at 5° C.

The results of this stability testing are presented in the table hereinbelow.

| Liquid fibrinogen | | | | |
|---|---|---|---|---|
| Minimum formulation | | | | |
| Stability testing at 5° C. | | T0 | T2M | T3M |
| Concentration (DO at 280 nm) | | 18.5 | 16.8 | 18.5 |
| pH | | 6.9 | 6.9 | 6.9 |
| Osmolality | | 209 | 207 | 210 |
| Coagulable fibrinogen | | 20 | 15.7 | 15.7 |
| Antigen fibrinogen | | 16.5 | 15.6 | 16.2 |
| Coagulable/antigen fibrinogen ratio | | 1.2 | 1.0 | 1.0 |
| SDS PAGE in | Aα1 (64 kDa) | 25.9 | 23.1 | 23.2 |
| reduced conditions | Aα2 (62 kDa) | 10.7 | 10.1 | 9.5 |
| | Aα3 (60 kDa) | 3.9 | 5.2 | 5.0 |
| | ΣAαₙ | 40.5 | 38.4 | 37.7 |
| | Bβ (54 kDa) | 30.8 | 31.1 | 31.2 |
| | γ′ (50 kDa) | 4.7 | 4.0 | 3.1 |
| | γ (48 kDa) | 23.9 | 26.5 | 27.9 |
| | Σγ | 28.6 | 30.5 | 31.0 |

The results obtained 2 and 3 months after the beginning of stability testing show that:
The results of SDS PAGE in reduced conditions show a low variation in the profiles of the alpha, beta and gamma chains that remain stable over time, confirming the retaining of all of the liquid fibrinogen;
The coagulable fibrinogen/antigen fibrinogen ratio is retained and stable at 1, confirming the retaining of all of and of the functionality of the liquid fibrinogen.

The results of this test clearly show that the fibrinogen composition is stable in liquid form at 5° C. for 3 months.

### Example 3: Identification of Anti-Fibrinogen Aptamers Used According to the Invention

### 1. Equipment and Method

Oligonucleotide Banks

The ssDNA bank used to carry out the SELEX method is constituted of a random region of 40 bases flanked by two constant primer regions of 18 bases.

Fibrinogen

The target protein is human fibrinogen. Various sources of human fibrinogen were used during the SELEX method:

Human fibrinogen: Two preparations of human fibrinogen were used and prepared in the form of a purified composition using human plasma that has a purity respectively of 95% and of 99.9%.

Transgenic fibrinogen: The transgenic fibrinogen was purified using transgenic cow milk up to a purity of 97%.

SELEX Protocol

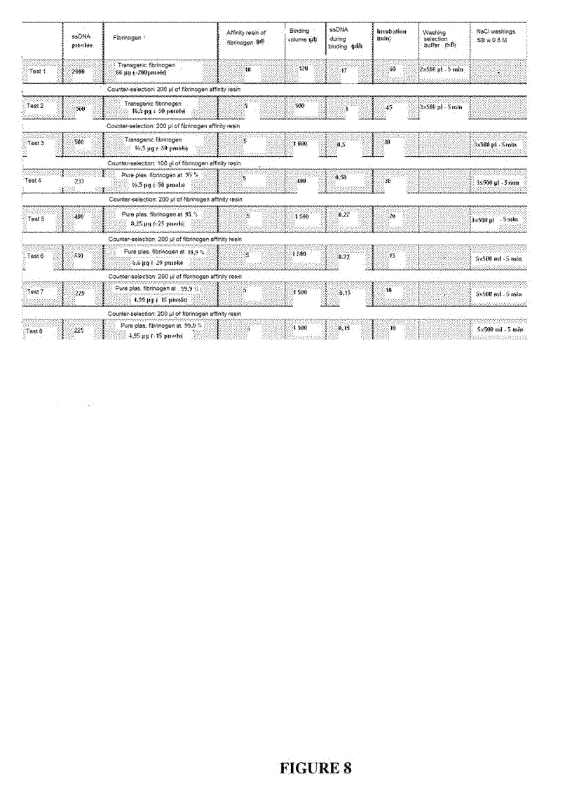

The fibrinogen (pure transgenic fibrinogen at 97% for tests 1 to 3 and the pure plasma fibrinogen at 95% for tests 4 and 5 and the pure plasma fibrinogen at 99.9% for tests 6 to 8) was immobilised on an affinity resin, while the quantity of target immobilised on the resin decreased continuously from tests 1 to 8 (see FIG. 8).

The immobilised target was incubated with a bank/pool of ssDNA at decreasing concentrations using a selection buffer (MOPS 50 mM, pH 6.30, NaCl 150 mM, MgCl₂ 5 mM) for a decreasing incubation time (see the table in FIG. 8).

The resin containing the fibrinogen/ssDNA was recovered and washed with the selection buffer during tests 1 and 2 and a wash buffer containing MOPS 50 mM, pH 6.30, NaCl 500 mM, MgCl₂ 5 mM of the tests 3 to 8 (see the table). After the washing, the bound ssDNA was eluted using an elution buffer (Tris-HCl 50 mM, pH 7.40, EDTA 200 mM). Before each test (except the first test), a step of selection of counter-ions was carried out by incubation of all the ssDNA with the affinity resin so as to prevent the enrichment of the anti-support aptamers. The parameters of the SELEX protocols are described in FIG. 8.

Determination of the Binding Affinity of the Aptamers by SPR

The chosen aptamer was synthesised with biotin and a spacer of triethylene glycol at the end 5′ of the oligonucleotide. A solution 1 μM of the aptamer was prepared using the SELEX selection buffer. The aptamer solution was heated to 90° C. for 5 min, incubated in ice for 5 min and balanced at the ambient temperature for 10 min. The preparation was injected into a sensor chip coated with streptavidin SA from Biocore T200 instrument (GE Healthcare) at a debit of 10 μl/ml for 7 min. Then, various concentrations of the target were injected onto the immobilised aptamer at a rate of 30 μl/min for 1 minute. After a dissociation for 1-2 min, a step of washing was carried out via injection of a suitable wash buffer at a rate of 30 μl/min for 1 min. For the elution, a suitable elution buffer was injected at a rate of 30 μl/min for 1-2 min. Finally, the sensor chip was regenerated via an injection of NaOH 50 mM at a rate of 30 μl/min for 30 s. During the experiment, the response signal was recorded in a sensorgram.

### 2. Results

The SELEX method made it possible to identify 67 anti-fibrinogen aptamers candidates, among which aptamers of SEQ ID NO 1, SEQ ID NO 58, SEQ ID NO 60 and 65 which has a strong affinity both for the human plasma and transgenic fibrinogen.

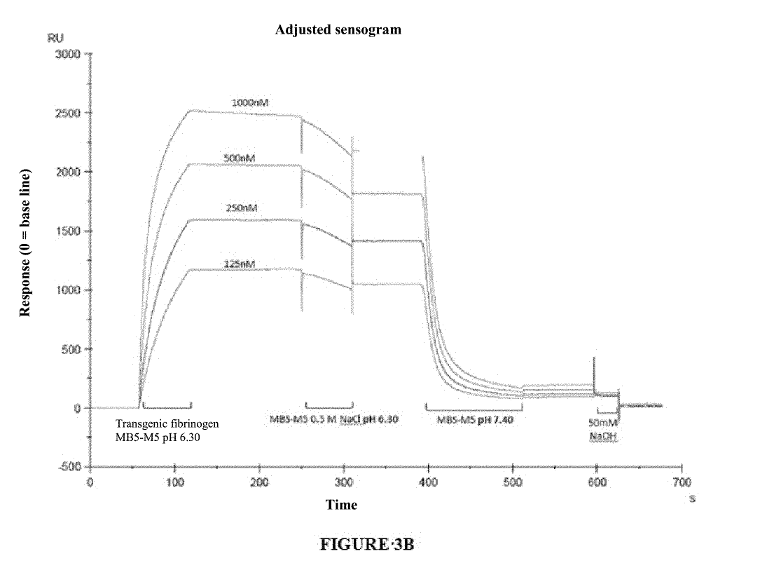

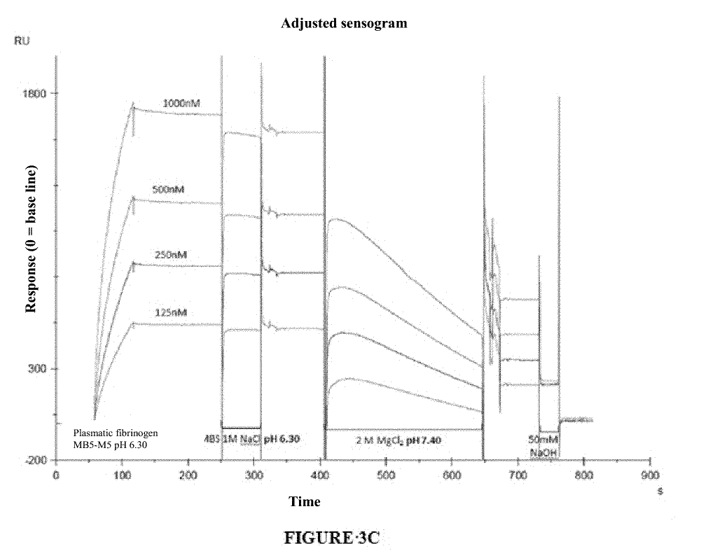

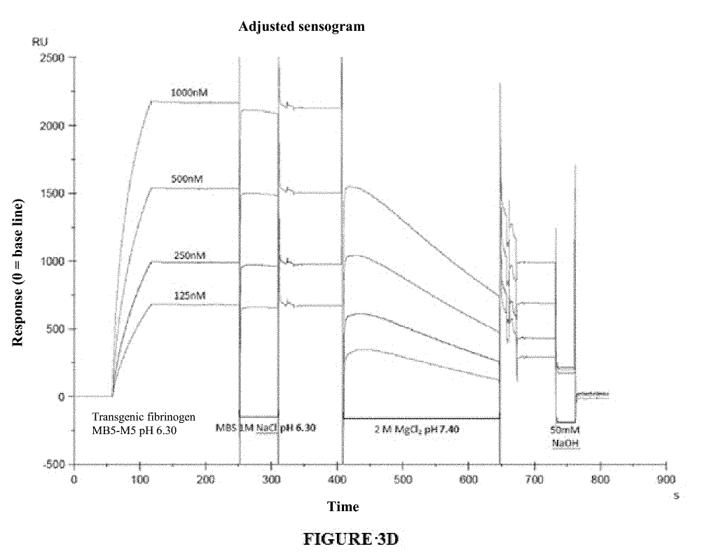

These aptamers bind to the human fibrinogen in a pH-dependent manner. The base sequences of aptamers of SEQ ID NO 1 and of SEQ ID NO 58 (namely the minimum sequence binding to the fibrinogen) were determined. The aptamer of SEQ ID NO 66 corresponds to the base sequence of the aptamer of SEQ ID NO 1. The aptamer of SEQ ID NO 67 corresponds to the base sequence of the aptamer of SEQ ID NO 58. FIGS. 3A-3D show the binding profile obtained for the base sequences of the aptamers of SEQ ID NO 1 and NO: 58 by SPR. The aptamers of SEQ ID NO 66 and 67 are able to bind specifically to the transgenic fibrinogen and to the plasma fibrinogen at pH 6.3 in a dose-dependent manner, as shown by the increase in the signals when the concentration in fibrinogen increases. The complex between the aptamers and the fibrinogen was not significantly dissociated by the increase in the concentration of NaCl. On the other hand, the injection of a buffer at pH 7.4 made it possible to dissociate the complex between the aptamers and, therefore, the fibrinogen was eluted (FIGS. 3A-3D).

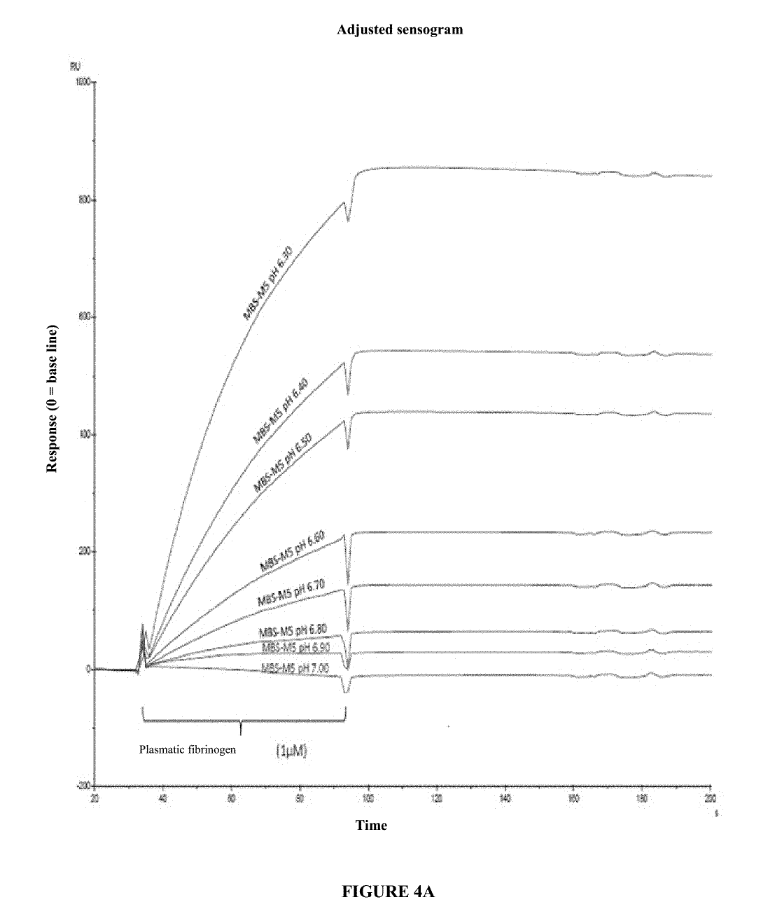

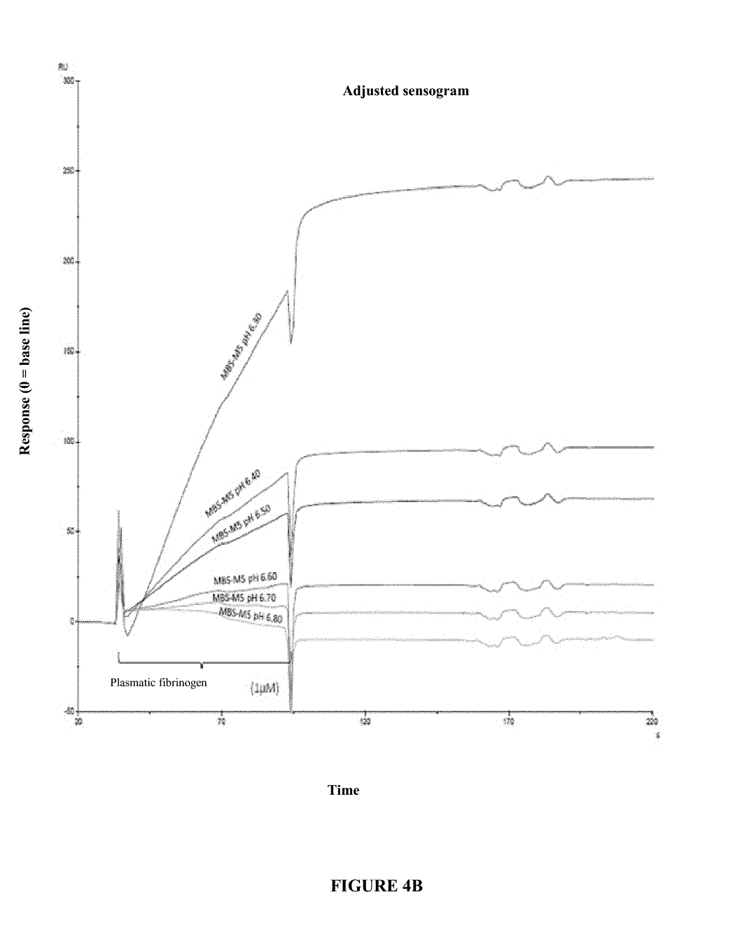

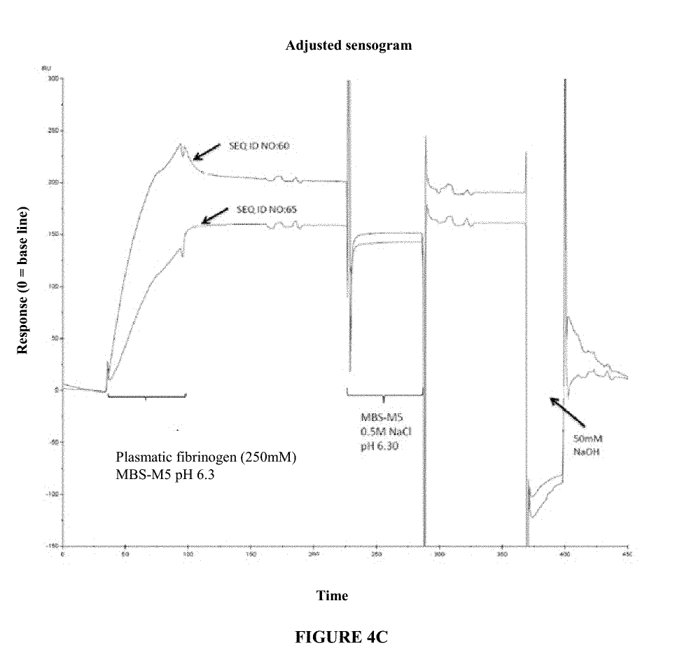

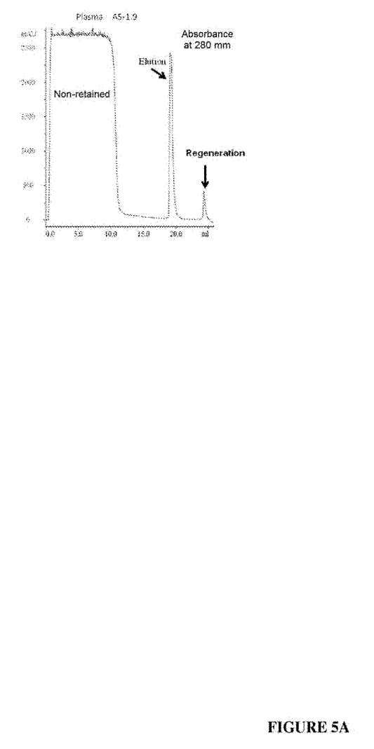

Indeed, the aptamers obtained by the method of the invention bind to the fibrinogen in a pH-dependent manner. This result is shown in FIGS. 4A and 4B respectively for aptamers of SEQ ID NO 66 and SEQ ID NO 67. The binding level of the fibrinogen decreases when the pH increases. The highest binding was observed at pH 6.3. The aptamers are not bound to the fibrinogen for a pH higher than 6.8.

### Example 4: Preparation of Affinity Supports for the Identified Aptamers

### 1. Equipment and Method

Affinity Supports

Two affinity supports were prepared by grafting of aptamers on the NHS-activated Sepharose (GE Healthcare). The first affinity support (affinity support No. 1) was prepared by grafting of aptamers of SEQ ID NO 66 (aptamer A5-1.9) comprising a spacer in C6 with an amino terminal group at its end 5′ and an inverted deoxy-thymidine at its end 3′. The second affinity support (affinity support No. 2) was prepared by grafting of aptamers of SEQ ID NO 67 (aptamer A5-2.9) comprising a spacer in C6 with an amino terminal group at its end 5′ and an inverted deoxy-thymidine at its end 3′.

1 volume of NHS-activated Sepharose gel placed in a column was rinsed with at least 10 volumes of solution of cold HCl 0.1 M, then balanced with at least 8 volumes of a solution of cold acetate 100 mM, pH 4.0.

After a centrifugation at 2000 g for 3 min, the supernatant is separated and the drained gel is re-suspended in 2 volumes of aptamer in a solution of acetate 100 mM pH 4.0. This suspension is incubated for 2 hours at ambient temperature under stirring.

Then, 1 volume of borate 200 mM pH 9 is added, this suspension is incubated at ambient temperature under stirring for 2 h 30.

After a centrifugation at 2000 g for 3 min, the supernatant is set aside. The drained gel is re-suspended in 2 volumes of a solution of Tris-HCl 0.1 M pH 8.5. The suspension is incubated at +4° C. under stirring for one night.

After an incubation and a centrifugation at 2000 g for 3 min, the supernatant is set aside. The gel is alternatively washed with 2 volumes of sodium acetate 0.1 M+NaCl 0.5% pH 4.2 and 2 volumes of a solution of Tris-HCl 0.1 M pH 8.5. This washing cycle is repeated once.

After a centrifugation at 2000 g for 3 min, the supernatant is separated. The drained gel is re-suspended in 2 volumes of a balancing buffer.

| | | Affinity | | Affinity | |
|---|---|---|---|---|---|
| | | support No. 1 | | support No. | |
| | | whereon | | 2 whereon | |
| | | aptamer | | aptamer | |
| | | groups of | | groups of | |
| | | SEQ ID | | SEQ ID | |
| | | NO 66 are | | NO 67 are | |
| | | grafted | | grafter | |
| Purification of the | Quantity of aptamer | 4.2 | mg | 2.5 | mg |
| fibrinogen | used for the grafting | | | | |
| from the Plasma | Volume of gel | 0.5 | mL | 0.5 | mL |
| | grafted | | | | |
| Purification of the | Quantity of aptamer | 174 | mg | 209 | mg |
| fibrinogen | used for the grafting | | | | |
| using a semi- | Volume of gel | 24 | mL | 42 | mL |
| purified fibrinogen | grafted | | | | |
| product | | | | | |

### Example 5: Purification of the Fibrinogen Using a Solution of Semi-Purified Fibrinogen on the Affinity Support of the Example 4

### 1. Material and Method

### —Conditions of the Affinity Chromatography

Affinity support no. 1: A solution of defrosted semi-purified fibrinogen (IP1: Fibrinogen intermediate product 1) obtained using human plasma was diluted 10 times in the binding buffer and the pH was adjusted to 6.3. The diluted IP1 was subjected to steps of chromatography on the support no 1. This step was repeated one time in order to obtain a sufficient quantity of fibrinogen for a step of ultrafiltration.

Affinity support no. 2: A solution of defrosted semi-purified fibrinogen (IP1: Fibrinogen intermediate product 1) obtained using human plasma was diluted 10 times in the binding buffer and the pH was adjusted to 6.3. The diluted IP1 was subjected to steps of chromatography on the support no 2. This step was repeated one time in order to obtain a sufficient quantity of fibrinogen for a step of ultrafiltration.

The conditions of the affinity chromatography are summarised for each affinity support:

| | Affinity | |
|---|---|---|
| | support No. 1 whereon | Affinity support No. 2 whereon |
| | aptamer groups of SEQ ID | aptamer groups of SEQ ID NO 67 |
| | NO 66 (A5-1.9) are grafted | (A5-2.9) are grafted |
| Binding | MOPS 50 mM, MgCl₂ | MOPS 50 mM, NaCl 150 mM, |
| buffer | 5 mM, NaCl 150 mM, | pH 6.3 |
| | pH 6.3 | |
| Wash | None | MOPS 50 mM, NaCl 2M, pH 7.4 |
| buffer | | |
| Elution | MOPS 50 mM, NaCl | MOPS 50 mM, MgCl₂ 2M, |
| buffer | 150 mM, pH 7.4 | pH 7.4 |

For each affinity support, the fibrinogen was eluted in moderate conditions by modification of the composition of the buffer. For the two chromatographies on the affinity supports no. 1 and no. 2, 2 fractions of eluate were produced and grouped together for a step of ultrafiltration.

### —Conditions of the Ultrafiltration

For each affinity support, a set of eluate fractions was subjected to an ultrafiltration at 100 kDa so as to concentrate the fibrinogen and to formulate it in sodium citrate 10 mM, argiline at a rate of 20 g/L at pH 7.4.

### —Analytical Methods

| Proteins | Titration methods |
|---|---|
| Fibronectin, Antigen fibrinogen | Nephelometry |
| Factor II, Factor XI, Factor XIII, Plasminogen | Elisa |
| Coagulation activity of the fibrinogen | Coagulation test |
| | (Von Clauss method) |

### 2. Results

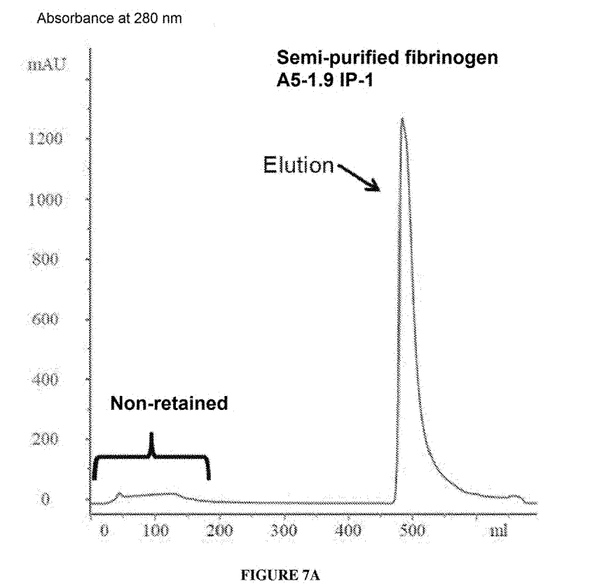

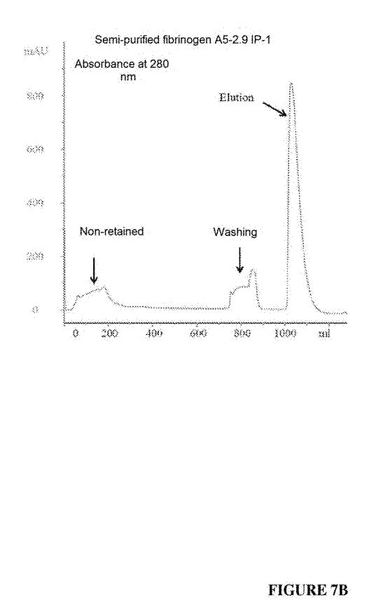

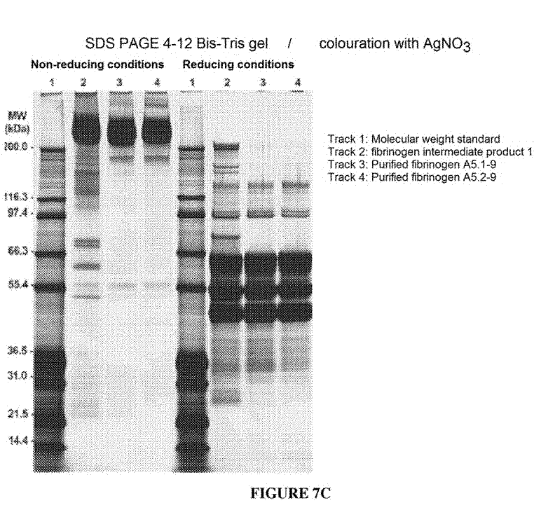

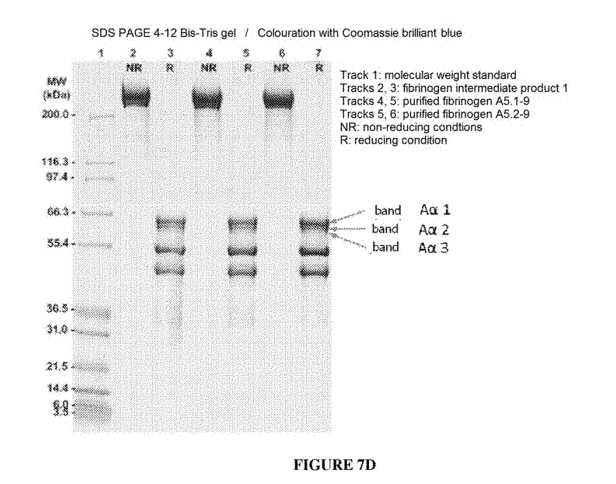

The results are shown in FIGS. 7A-7B and 7C-7D. FIGS. 7A and 7B show the chromatography profile obtained for the purification of the fibrinogen using a semi-purified solution of fibrinogen respectively on affinity supports no. 1 and no. 2. Fibrinogen was eluted by raising the pH to 7.4 and by adding MgCl₂ for the affinity support no. 2 and by elimination of the Mg²⁺ for the affinity support no. 1. The analysis by electrophoresis of the fractions obtained by chromatography (FIGS. 7C and 7D) showed that contaminants present in the loaded material (IP1) are notably eliminated, with practically solely the fibrinogen being visible in the eluate. In addition, reducing conditions show that the fibrinogen in the eluate is a native form without visible degradation (Aα1 is the largest band among the bands Aα).

The outputs and the concentration in fibrinogen obtained are summarised in the table hereinbelow:

| | Affinity | Affinity |
|---|---|---|
| | support No. 1 | support No. 2 |
| Chromatography output (%) | 51 | 71 |
| Concentration of the antigen fibrinogen | 13.1 | 14.2 |
| obtained after ultrafiltration (mg/ml) | | |

The active fibrinogen is revealed by a ratio between the coagulant fibrinogen and the antigen fibrinogen close to 1. An analysis of the concentrated and formulated fibrinogen prepared with the two affinity supports is detailed in the following table:

| | | Coagulation/ |
|---|---|---|
| | Coagulation activity of the | antigen |
| | fibrinogen g/L | fibrinogen ratio |
| Starting material (IP1 | 17.6 | 1.17 |
| fibrinogen) | | |
| Purified fibrinogen | 13.9 | 1.06 |
| concentrate - Support no. 1 | | |
| Purified fibrinogen | 14.5 | 1.02 |
| concentrate - Support no. 2 | | |

For the two purified fibrinogens, the ratio between the coagulation and antigen fibrinogen was about 1.0 for the two aptamers. The moderate chromatography conditions allow for the preparation of the purified fibrinogen having a retained activity.

The table hereinbelow shows the titration of the contaminant proteins in the starting material and in the purified fraction of fibrinogen obtained with the affinity support of the invention:

| | Semi-purified | | | | | | | |
|---|---|---|---|---|---|---|---|---|
| | fibrinogen | | | | | | | |
| | (starting | | | | | | | |
| Contaminant | composition) | | Affinity support no. 1 | | | Affinity support no. 2 | | |
| proteins | Concentration | | Concentration | | Elimination | Concentration | | Elimination |
| Fibronectin | 0.55 | g/L | 0.02 | g/L | 96.3% | 0.02 | g/L | 95.1% |
| Factor II | 0.13 | mUI/mL | 0.03 | mUI/mL | 70.8% | 0.04 | mUI/mL | 66.3% |
| Factor XI | 21.0 | mUI/mL | 2.8 | mUl/mL | 83.8% | 3.6 | mUI/mL | 80.8% |
| Factor XIII | 10000 | mUI/mL | 10 | mUI/mL | 99.9% | 42 | mUI/mL | 99.5% |
| Plasminogen | 56 | μg/mL | 0.21 | μg/mL | 99.5% | 0.21 | μg/mL | 99.6% |

A good elimination of the contaminant proteins is obtained with an elimination ranging from 65% up to 99% of the starting material.

The chromatography conditions allowed for the elimination of more than 99.5% of the initial plasminogen, which is one of the most problematic contaminants from the standpoint of the stability of the fibrinogen.

The aptamers identified by SELEX are suitable for a use as an affinity ligand in the purification of fibrinogen by chromatography. It should be noted that the aptamers identified by the method of the invention allow for the selective binding and then the elution of the fibrinogen in moderate and non-denaturing conditions.

### Example 6: Purification of Fibrinogen by Chromatography Using Plasma

Affinity support No. 1: The plasma was defrosted, filtered at 0.45 μm, diluted 10 times with the binding buffer and then the pH was adjusted to 6.3. A diluted solution was subjected to steps of chromatography on the support No. 1.

Affinity support No. 2: The plasma was defrosted, filtered at 0.45 μm, diluted 10 times with the binding buffer and then the pH was adjusted to 6.3. A diluted solution was subjected to steps of chromatography on the support No. 2.

The conditions of the affinity are summarised, for each affinity support, in the table hereinbelow:

| | Affinity support No. 1 whereon | Affinity support No. 1 whereon |
|---|---|---|
| | aptamer groups of SEQ ID NO 66 | aptamer groups of SEQ ID NO 67 |
| | (A5-1.9) are grafted | (A5-2.9) are grafted |
| Binding buffer | MOPS 50 mM, MgCl₂ 5 mM, | MOPS 50 mM, NaCl 150 mM, |
| | NaCl 150 mM, pH 6.3 | pH 6.3 |
| Wash buffer | Return to the base line with the | MOPS 50 mM, NaCl 2M, pH 7.4 |
| | binding buffer | |
| Elution buffer | MOPS 50 mM, NaCl 150 mM, | MOPS 50 mM, MgCl₂ 2M, |
| | pH 7.4 | pH 7.4 |
| Regeneration | MOPS 50 mM, MgCl₂ 2M, | identical to the elution buffer |
| buffer | pH 7.4 | |

Pour each affinity support, the fibrinogen was eluted in moderate conditions by modification of the composition of the buffer.

### 2. Results

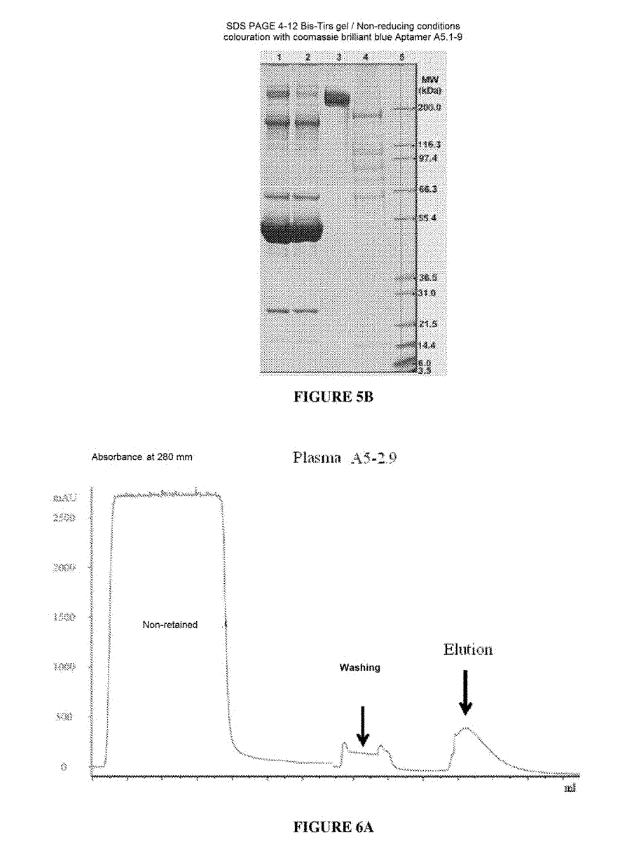

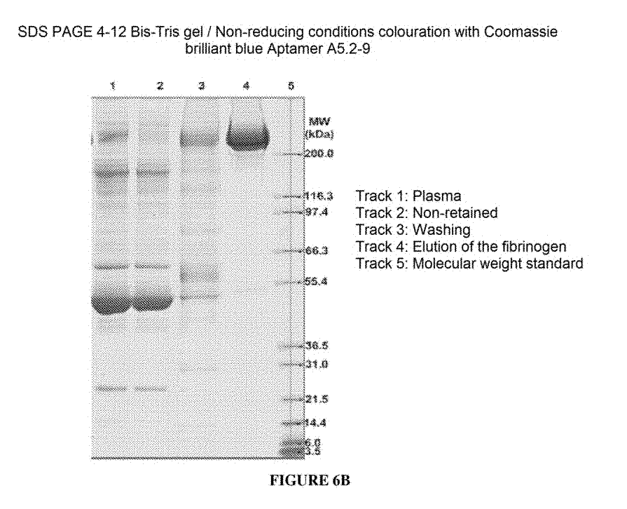

The results are shown in FIGS. 5A-5B and 6A-6B. FIGS. 5A and 6A show the chromatography profile obtained for the purification of the fibrinogen using plasma respectively on the affinity supports No. 1 and No. 2. It should be noted that most of the contaminant proteins were not retained on the stationary phase while the fibrinogen is bound to the support. The fibrinogen was eluted by raising the pH to 7.4 and by adding MgCl₂ 2 M pour the affinity support No. 2 and by elimination of the Mg²⁺ for the affinity support No. 1. The analysis by electrophoresis of the fractions obtained by chromatography (FIG. 5B and FIG. 6B) showed that the fibrinogen was mainly present in the elution fraction while the contaminant proteins were present in the non-retained fraction, in the wash fraction or in the regeneration fraction. Indeed, the elution fractions migrated in form of a single band. The relative purity (determined by SDS PAGE) of the fractions of eluate fibrinogen was higher than 95%.

These results reveal that the aptamers used according to the invention are particularly suitable for a use as affinity ligands in the purification of the fibrinogen using complex starting compositions.

### Example 7: Optimisation of the Base Sequence of SEQ ID NO: 66

### 1. Material and Method

—Preparation of variants of SEQ ID NO: 66:

In the first test, 28 variants were designed by systematic elimination of 2 consecutive nucleotides per variant (1/2, 3/4, 5/6, etc.). In the following test, combinations of deletions that did not lead to a loss of affinity were combined.

### —Competitive Binding Test

In order to compare the affinity of the designed variants with that of the parental aptamer, a concurrent test was conducted. Firstly, a 1 μM solution of the aptamer of SEQ ID NO 66 was prepared using binding buffer. The aptamer solution was heated to 90° C. for 5 min, incubated in ice for 5 min and balanced at the ambient temperature for 10 min. The preparation was injected into a sensor chip coated with streptavidin SA from Biacore T200 instrument (GE Healthcare) at a rate of 10 μl/min for 7 min. Then, mixtures containing a variant (2 μM) and the human plasma fibrinogen (0.4 μM) were injected on the aptamer immobility at a rate of 30 μl/min for 1 minute. The response obtained for the various fragment/fibrinogen mixtures was compared.

### 2. Results

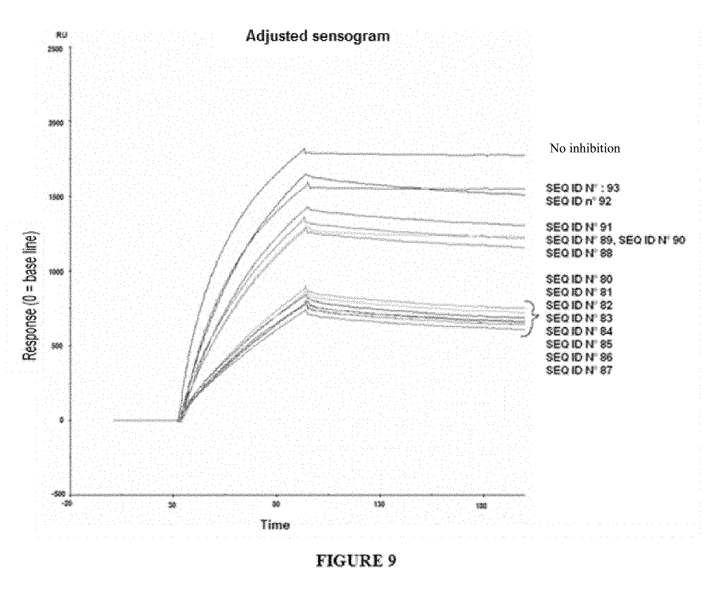

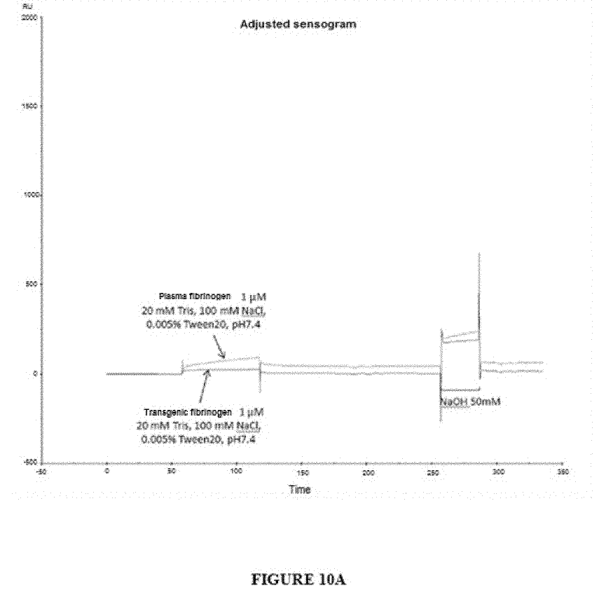

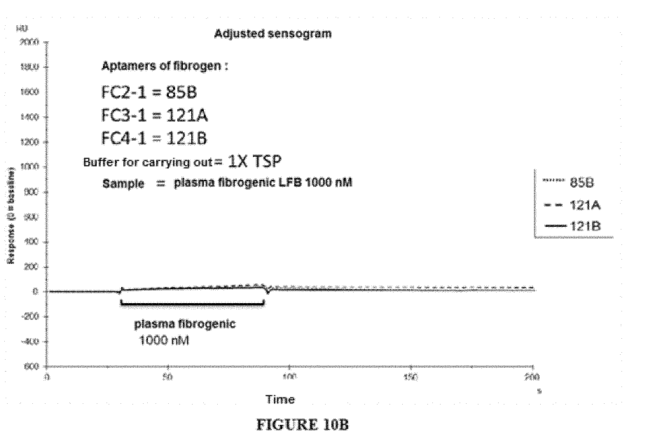

As shown in FIG. 9, variants of the sequence of SEQ ID NO: 80-93 have a significant inhibition of the binding signal and consequently they have a considerable affinity for the fibrinogen. The highest affinity was observed for the variants of SEQ ID NO: 80-87 containing deletions at the positions 01/02, at the positions 01/02/19/20/21, at the positions 01/02/14/15/16/20/21/22, at the positions 01/02/18/19/20/21, at the positions 01/02/18/19/20, at the positions 01/02/15/16/20/21, and at the positions 01/02/19/20, respectively.

### Example 8: Stability of a Composition of Liquid Fibrinogen

### Composition of Fibrinogen

The fibrinogen composition was obtained using chromatography support no. 1 implementing the aptamer A5-1.9 (SEQ ID NO 66) is formulated at least with arginine 100 mM and with citrate 8.5 mM at pH 7.0.

### Stability of the Fibrinogen Composition

The composition is placed into stability, under air (in the open air), without stirring, in a cold room at +5° C.

Samples are taken before stability testing (TO) and one month after the beginning of stability testing (TIM).

### Results of Stability Testing at 5° C.

The results of this stability test are presented in the table hereinbelow.

| Liquid fibrinogen (purified by A5-1.9) | | |
|---|---|---|
| Minimum formulation | | |
| Stability testing at 5° C. | T0 | T1M |
| pH | 6.9 | 7.0 |
| Osmolality | 206 | 204 |
| Coagulable fibrinogen | 15.1 | 14.4 |
| Antigen fibrinogen | 14.2 | 13.4 |
| Coagulable/antigen fibrinogen ratio | 1.06 | 1.07 |

| | | | |
|---|---|---|---|
| SDS PAGE in | Aα1 (64 kDa) | 23.7 | 25.7 |
| reduced conditions | Aα2 (62 kDa) | 11.7 | 10.4 |
| | Aα3 (60 kDa) | 3.7 | 2.3 |
| | ΣAαₙ | 39.1 | 38.4 |
| | Bβ (54 kDa) | 31.2 | 31.9 |
| | γ′ (50 kDa) | 6 | 5.6 |
| | γ (48 kDa) | 23.7 | 24.1 |
| | Σγ | 29.7 | 29.7 |

The results after one month of stability testing show that:
The results of SDS PAGE in reduced conditions show a low variation in the profiles of the alpha, beta and gamma chains that remain stable over time, confirming the retaining of all of the liquid fibrinogen;
The coagulable fibrinogen/antigen fibrinogen ratio is retained and stable at 1, confirming the retaining of all of and of the functionality of the liquid fibrinogen.

The results of this test clearly show that the fibrinogen composition is stable in liquid form at 5° C. for 1 month.

Table of sequences

| SEQ ID NO | Description |
|---|---|
| 1-57 | Aptamers of the first subgroup |
| 58-65 | Aptamers of the second subgroup |
| 66 | Basic sequence of the aptamer of SEQ ID NO: 1 (A.5.1.9) |
| 67 | Basic sequence of the aptamer of SEQ ID NO: 58 (A.5.2.9) |
| 68-74 | Central regions of SEQ ID NO: 59-65 |
| 75 | First primer sequence |
| 76 | Second primer sequence |
| 77-79 | Nucleotide groups present in the consensus sequence of |
| | the second subgroup of aptamers |
| 80-93 | Variants of the aptamer of SEQ ID N^{o}: 66 |
| 94 | Central region of SEQ ID NO: 1 |
| 95 | Central region of SEQ ID NO: 58 |

1-22. (canceled)

23. Composition comprising stable fibrinogen in liquid form, wherein said composition has a coagulable fibrinogen/fibrinogen antigen ratio higher than 0.5.

24. Composition according to claim 23, wherein said composition is stable for at least 3 months at 4° C.

25. Composition according to claim 23, wherein said composition has a quantity of fibrinogen monomers retained during stability testing higher than 50% of the initial rate of fibrinogen monomers.

26. Composition according to claim 23, wherein said composition has a quantity of polymers formed during stability testing lower than 10% of the initial rate of fibrinogen polymers.

27. Composition according to claim 23, wherein:
all of the alpha chains are retained at at least 50%, and/or
all of the beta chains are retained at at least 50%, and/or
all of the gamma chains are retained at at least 50%.

28. Composition according to claim 23, wherein the turbidity measured after the stability test is lower than 130% of the turbidity measured before the stability test.

29. Composition according to claim 23, wherein said composition is devoid of proteases and/or of activators of fibrinolysis.

30. Composition according to claim 23, wherein said composition is devoid of protease inhibitors and/or of anti-fibrinolytics.

31. Composition according to claim 23, wherein said composition further comprises pharmaceutically acceptable excipients.

32. Composition according to claim 23, wherein said pharmaceutically acceptable excipients consist of arginine and of citrate.

33. Composition according to claim 23, constituted of fibrinogen, arginine and citrate.

34. Composition according to claim 23, wherein the composition is devoid of isoleucine and/or of glycine.

35. Composition according to claim 23, wherein the composition is devoid of albumin.

36. Composition according to claim 23, wherein the composition is devoid of metal ions, in particular calcium.

37. Composition comprising fibrinogen according to claim 23, said composition not having been subjected to any prior step of freeze-drying, desiccation, dehydration or drying.

38. Composition comprising fibrinogen according to claim 23, said composition not having been subjected to any prior step of reconstitution of a lyophilisate.

39. Composition comprising fibrinogen according to claim 23, able to be obtained by the method comprising the following steps:
a step of purification by affinity chromatography,
at least one step of biological security, and
a step of formulation in liquid form.

40. Composition according to claim 39, wherein the affinity chromatography consists of chromatography using ligands type moiety derived from llama antibody or aptamers.

41. Composition comprising fibrinogen according to claim 23 able to be obtained according to the method comprising the following steps:
purification of a plasma or of a cryosupernatant fraction of blood plasma by separation by affinity gel chromatography,
recovery of the purified absorbed fraction comprising fibrinogen,
adding pharmaceutically acceptable excipients.

42. Composition according to claim 23 for use in therapy.

43. Composition for use according to claim 42, wherein said composition is suitable for being administered intravenously.


SEQUENCE LISTING (95 sequences)

SEQ ID NO: 1 - DNA, 76 bp, Artificial
  Aptamer
gggtcaatgccaggtctcggacctggaatccgccacccgcattagaaccagggttgacatcggctcgcaagcagtc

SEQ ID NO: 2 - DNA, 76 bp, Artificial
  Aptamer
gggtcaatgccaggtctcggacctggaatccgccacccgcactagaaccagggttgacatcggctcgcaagcagtc

SEQ ID NO: 3 - DNA, 75 bp, Artificial
  Aptamer
gggtcaatgccaggtctcggacctggaatccgccacccgcattagaaccagggttgacatcggctcgcagcagtc

SEQ ID NO: 4 - DNA, 74 bp, Artificial
  Aptamer
gggtcaatgccaggtctcggacctggaatccgccacccgcattagaaccagggttgacatcggctcgcgcagtc

SEQ ID NO: 5 - DNA, 75 bp, Artificial
  Aptamer
gggtcaatgccaggtctcggacctggaatccgccacccgcattagaaccagggttgacatcggctcgcaagcagc

SEQ ID NO: 6 - DNA, 76 bp, Artificial
  Aptamer
gggtcaatgccaggtctcggacctggaatccgccacccgcattagaaccagggttgacgtcggctcgcaagcagtc

SEQ ID NO: 7 - DNA, 76 bp, Artificial
  Aptamer
gggtcaatgccaggtctcagacctggaatccgccacccgcattagaaccagggttgacatcggctcgcaagcagtc

SEQ ID NO: 8 - DNA, 76 bp, Artificial
  Aptamer
gggtcaatgccaggtctcggacctggaatccgccacccgcattaaaaccagggttgacatcggctcgcaagcagtc

SEQ ID NO: 9 - DNA, 76 bp, Artificial
  Aptamer
gggtcaatgccaggtctcggacctggaatccgtcacccgcattagaaccagggttgacatcggctcgcaagcagtc

SEQ ID NO: 10 - DNA, 76 bp, Artificial
  Aptamer
gggtcaatgccaggtctcggacctggaatccgccacccgcattagaaccagggttgacatcggctcgcaagcggtc

SEQ ID NO: 11 - DNA, 76 bp, Artificial
  Aptamer
gggtcaatgccaggtctcggacccggaatccgccacccgcattagaaccagggttgacatcggctcgcaagcagtc

SEQ ID NO: 12 - DNA, 76 bp, Artificial
  Aptamer
gggtcaatgccaggtctcggacctggaatccgccacccgcattagaaccagggttgacatcgactcgcaagcagtc

SEQ ID NO: 13 - DNA, 75 bp, Artificial
  Aptamer
gggtcaatgccaggtctcggacctggaatccgccacccgcactagaaccagggttgacatcggctcgcaagcatc

SEQ ID NO: 14 - DNA, 75 bp, Artificial
  Aptamer
gggtcaatgccaggtctcggacctggaatccgccacccgcactagaaccagggttgacatcggctcgcaagcgtc

SEQ ID NO: 15 - DNA, 76 bp, Artificial
  Aptamer
gggtcaatgccaggtctcggacccggaatccgccacccgcactagaaccagggttgacatcggctcgcaagcagtc

SEQ ID NO: 16 - DNA, 76 bp, Artificial
  Aptamer
gggtcaatgccaggtctcggaccaggaatccgccacccgcactagaaccagggttgacatcggctcgcaagcagtc

SEQ ID NO: 17 - DNA, 75 bp, Artificial
  Aptamer
gggtcaatgccaggtctcggacctggaatccgccacccgcactagaaccagggttgacatcggctgcaagcagtc

SEQ ID NO: 18 - DNA, 76 bp, Artificial
  Aptamer
gggtcaatgccaggtctcggacctggaatccgccacccgcactagagccagggttgacatcggctcgcaagcagtc

SEQ ID NO: 19 - DNA, 76 bp, Artificial
  Aptamer
gggtcaatgccaggtctcggacctggaatccgccacccgcactagaaccagggttgacatcggctcacaagcagtc

SEQ ID NO: 20 - DNA, 75 bp, Artificial
  Aptamer
gggtcaatgccaggtctcggacctggaatccgccacccgcactagaaccagggttgacatcggctccaagcagtc

SEQ ID NO: 21 - DNA, 74 bp, Artificial
  Aptamer
gggtcaatgccaggtctcggacctggaatccgccacccgcactagaaccagggttgacatcggctcaagcagtc

SEQ ID NO: 22 - DNA, 76 bp, Artificial
  Aptamer
gggtcaatgccaggtctcggacctggaatccgccacccgcactagaaccagggttgacatcggctcggaagcagtc

SEQ ID NO: 23 - DNA, 75 bp, Artificial
  Aptamer
gggtcaatgccaggtctcggacctggaatccgccacccgcactagaaccagggttgacatcggctcgaagcagtc

SEQ ID NO: 24 - DNA, 75 bp, Artificial
  Aptamer
gggtcaatgccaggtctcggacctggaatccgccacccgcactagaaccagggttgacatcggctcgcagcagtc

SEQ ID NO: 25 - DNA, 74 bp, Artificial
  Aptamer
gggtcaatgccaggtctcggacctggaatccgccacccgcactagaaccagggttgacatcggctcgcgcagtc

SEQ ID NO: 26 - DNA, 76 bp, Artificial
  Aptamer
gggtcaatgccaggtctcggacctggaatccgccacccgcactagaaccagggttgacatgggctcgcaagcagtc

SEQ ID NO: 27 - DNA, 74 bp, Artificial
  Aptamer
gggtcaatgccaggtctcggacctggaatccgccacccgcactagaaccagggttgacatcggctcgcacagtc

SEQ ID NO: 28 - DNA, 75 bp, Artificial
  Aptamer
gggtcaatgccaggtctcggacctggaatccgccacccgcactagaaccagggttgacatcggctcgcaacagtc

SEQ ID NO: 29 - DNA, 76 bp, Artificial
  Aptamer
gggtcaatgccaggtctcggacctggaatccgccacccgcactagaaccagggttgacatcagctcgcaagcagtc

SEQ ID NO: 30 - DNA, 76 bp, Artificial
  Aptamer
gggtcaatgccaggtctcggacctggaatccgccacccgcactagaaccagggctgacatcggctcgcaagcagtc

SEQ ID NO: 31 - DNA, 76 bp, Artificial
  Aptamer
gggtcaatgccaggtctcggacctggaatccgccacccgcattagaaccagggctgacatcggctcgcaagcagtc

SEQ ID NO: 32 - DNA, 76 bp, Artificial
  Aptamer
gggtcaatgccaggtctcggacctggaatccgccacccgcactagaaccagggttggcatcggctcgcaagcagtc

SEQ ID NO: 33 - DNA, 76 bp, Artificial
  Aptamer
gggtcaatgccaggtctcggacctggaatccgccacccgcattagaaccagggttggcatcggctcgcaagcagtc

SEQ ID NO: 34 - DNA, 76 bp, Artificial
  Aptamer
gggtcaatgccaggtctcggacctgggatccgccacccgcactagaaccagggttgacatcggctcgcaagcagtc

SEQ ID NO: 35 - DNA, 76 bp, Artificial
  Aptamer
gggtcaatgccaggtctcggacctgggatccgccacccgcattagaaccagggttgacatcggctcgcaagcagtc

SEQ ID NO: 36 - DNA, 76 bp, Artificial
  Aptamer
gggtcaatgccaggtctcggacctggaatccgccacccgcactagaaccagggttgacatcgggtcgcaagcagtc

SEQ ID NO: 37 - DNA, 76 bp, Artificial
  Aptamer
gggtcaatgccaggtctcggacctggaatccgccacccgcattagaaccagggttgacatcgggtcgcaagcagtc

SEQ ID NO: 38 - DNA, 76 bp, Artificial
  Aptamer
gggtcaatgccaggtctcggacctggaatccgccacccgcattagaaccagggtcgacatcggctcgcaagcagtc

SEQ ID NO: 39 - DNA, 76 bp, Artificial
  Aptamer
gggtcaatgccaggtctcggacctggaatccgccacccgcactagaaccagggtcgacatcggctcgcaagcagtc

SEQ ID NO: 40 - DNA, 76 bp, Artificial
  Aptamer
gggtcaatgccaggtctcgggcctggaatccgccacccgcattagaaccagggttgacatcggctcgcaagcagtc

SEQ ID NO: 41 - DNA, 76 bp, Artificial
  Aptamer
gggtcaatgccaggtctcgggcctggaatccgccacccgcactagaaccagggttgacatcggctcgcaagcagtc

SEQ ID NO: 42 - DNA, 76 bp, Artificial
  Aptamer
gggtcaatgccaggtctcggacctggaatccgccgcccgcattagaaccagggttgacatcggctcgcaagcagtc

SEQ ID NO: 43 - DNA, 76 bp, Artificial
  Aptamer
gggtcaatgccaggtctcggacctggaatccgccgcccgcactagaaccagggttgacatcggctcgcaagcagtc

SEQ ID NO: 44 - DNA, 76 bp, Artificial
  Aptamer
gggtcaatgccaggtctcggacctggaatccgcctcccgcattagaaccagggttgacatcggctcgcaagcagtc

SEQ ID NO: 45 - DNA, 76 bp, Artificial
  Aptamer
gggtcaatgccaggtctcggacctggaatccgccacccgcattagaaccagggttgacatcggctggcaagcagtc

SEQ ID NO: 46 - DNA, 74 bp, Artificial
  Aptamer
gggtcaatgccaggtctcggacctggaatccgccacccgcattagaaccagggttgacatcggctcaagcagtc

SEQ ID NO: 47 - DNA, 76 bp, Artificial
  Aptamer
gggtcaatgccaggtctcggacctggaatccgccacccgcattagaaccagggttgacatcggctcacaagcagtc

SEQ ID NO: 48 - DNA, 75 bp, Artificial
  Aptamer
gggtcaatgccaggtctcggacctggaatccgccacccgcattagaaccagggttgacatcggctcgaagcagtc

SEQ ID NO: 49 - DNA, 76 bp, Artificial
  Aptamer
gggtcaatgccaggtctcggacctggaatccgccacccgcattagaaccagggttgacatcggctcggaagcagtc

SEQ ID NO: 50 - DNA, 76 bp, Artificial
  Aptamer
gggtcaatgccaggtctcggacctggaatccgccacccgcattagaaccagggttgacatcggctcgcaaggagtc

SEQ ID NO: 51 - DNA, 76 bp, Artificial
  Aptamer
gggtcaatgccaggtctcggacctggaatccaccacccgcattagaaccagggttgacatcggctcgcaagcagtc

SEQ ID NO: 52 - DNA, 76 bp, Artificial
  Aptamer
gggtcaatgccaggtctcggacctggaatccgccacccgcattagaaccggggttgacatcggctcgcaagcagtc

SEQ ID NO: 53 - DNA, 75 bp, Artificial
  Aptamer
gggtcaatgccaggtctcggacctggaatccgccacccgcattagaaccagggttgacatcggctcgcaacagtc

SEQ ID NO: 54 - DNA, 76 bp, Artificial
  Aptamer
gggtcaatgccaggtctcgaacctggaatccgccacccgcattagaaccagggttgacatcggctcgcaagcagtc

SEQ ID NO: 55 - DNA, 76 bp, Artificial
  Aptamer
gggtcaatgccaggtctcgtacctggaatccgccacccgcattagaaccagggttgacatcggctcgcaagcagtc

SEQ ID NO: 56 - DNA, 76 bp, Artificial
  Aptamer
gggtcaatgccaggtctcggacctggaatccgccacccgcattagatccagggttgacatcggctcgcaagcagtc

SEQ ID NO: 57 - DNA, 76 bp, Artificial
  Aptamer
gggtcaatgccaggtctcggacctggaatccgccacccgcattaggaccagggttgacatcggctcgcaagcagtc

SEQ ID NO: 58 - DNA, 76 bp, Artificial
  Aptamer
gggtcaatgccaggtctcaactttcgcgtgtggttggtagggctaggtgtatacgcatatcggctcgcaagcagtc

SEQ ID NO: 59 - DNA, 75 bp, Artificial
  Aptamer
gggtcaatgccaggtctcaggtgagtcttacggatgtggttggtagggctaggtgtatatcggctcgcaagcagt

SEQ ID NO: 60 - DNA, 76 bp, Artificial
  Aptamer
gggtcaatgccaggtctctgcgtacttatctcgatgtggttggtagggataggtgtatatcggctcgcaagcagtc

SEQ ID NO: 61 - DNA, 74 bp, Artificial
  Aptamer
gggtcaatgccaggtctcgatctctgtggttggtaggggagtgtgtatagtgtcgtatcggctcgcaagcagtc

SEQ ID NO: 62 - DNA, 75 bp, Artificial
  Aptamer
gggtcaatgccaggtctcgctgtggttggtagggataggtgtatggcagctggataggatcggctcgcaagcagt

SEQ ID NO: 63 - DNA, 75 bp, Artificial
  Aptamer
gggtcaatgccaggtctcaataaaagtagctgatgtgcgttggtagggaacggtgtatatcggctcgcaagcagt

SEQ ID NO: 64 - DNA, 75 bp, Artificial
  Aptamer
gggtcaatgccaggtctcggcgtagaagccggaatgtggttggtagggttaggtgtatatcggctcgcaagcagt

SEQ ID NO: 65 - DNA, 76 bp, Artificial
  Aptamer
gggtcaatgccaggtctcgagccggtgttgtgttatgttggtagggaaggtgtataatatcggctcgcaagcagtc

SEQ ID NO: 66 - DNA, 58 bp, Artificial
  Core sequence A.5-1.9
gggtcaatgccaggtctcggacctggaatccgccacccgcattagaaccagggttgac

SEQ ID NO: 67 - DNA, 34 bp, Artificial
  Core sequence A5-2.9
cgcgtgtggttggtagggctaggtgtatacgcat

SEQ ID NO: 68 - DNA, 40 bp, Artificial
  Central region
aggtgagtcttacggatgtggttggtagggctaggtgtat

SEQ ID NO: 69 - DNA, 40 bp, Artificial
  Central region
tgcgtacttatctcgatgtggttggtagggataggtgtat

SEQ ID NO: 70 - DNA, 38 bp, Artificial
  Central region
gatctctgtggttggtaggggagtgtgtatagtgtcgt

SEQ ID NO: 71 - DNA, 40 bp, Artificial
  Central region
gctgtggttggtagggataggtgtatggcagctggatagg

SEQ ID NO: 72 - DNA, 40 bp, Artificial
  Central region
aataaaagtagctgatgtgcgttggtagggaacggtgtat

SEQ ID NO: 73 - DNA, 40 bp, Artificial
  Central region
ggcgtagaagccggaatgtggttggtagggttaggtgtat

SEQ ID NO: 74 - DNA, 40 bp, Artificial
  Central region
gagccggtgttgtgttatgttggtagggaaggtgtataat

SEQ ID NO: 75 - DNA, 18 bp, Artificial
  Primer
gggtcaatgccaggtctc

SEQ ID NO: 76 - DNA, 18 bp, Artificial
  Primer
atcggctcgcaagcagtc

SEQ ID NO: 77 - DNA, 10 bp, Artificial
  consensus moiety
gttggtaggg

SEQ ID NO: 78 - DNA, 7 bp, Artificial
  Consensus moiety
ggtgtat

SEQ ID NO: 79 - DNA, 3 bp, Artificial
  consensus moiety
tgt

SEQ ID NO: 80 - DNA, 56 bp, Artificial
  Aptamer
gtcaatgccaggtctcggacctggaatccgccacccgcattagaaccagggttgac

SEQ ID NO: 81 - DNA, 53 bp, Artificial
  Aptamer
gtcaatgccaggtctccctggaatccgccacccgcattagaaccagggttgac

SEQ ID NO: 82 - DNA, 50 bp, Artificial
  Aptamer
gtcaatgccagtcgctggaatccgccacccgcattagaaccagggttgac

SEQ ID NO: 83 - DNA, 52 bp, Artificial
  Aptamer
gtcaatgccaggtctcctggaatccgccacccgcattagaaccagggttgac

SEQ ID NO: 84 - DNA, 52 bp, Artificial
  Aptamer
gtcaatgccaggtcacctggaatccgccacccgcattagaaccagggttgac

SEQ ID NO: 85 - DNA, 53 bp, Artificial
  Aptamer
gtcaatgccaggtcgacctggaatccgccacccgcattagaaccagggttgac

SEQ ID NO: 86 - DNA, 52 bp, Artificial
  Aptamer
gtcaatgccaggtcgcctggaatccgccacccgcattagaaccagggttgac

SEQ ID NO: 87 - DNA, 54 bp, Artificial
  Aptamer
gtcaatgccaggtctcacctggaatccgccacccgcattagaaccagggttgac

SEQ ID NO: 88 - DNA, 54 bp, Artificial
  Aptamer
gtcaatgccaggtcggacctggaatccgccacccgcattagaaccagggttgac

SEQ ID NO: 89 - DNA, 55 bp, Artificial
  Aptamer
gtcaatgccaggtctcggacctggaatccgccacccgcattagaaccagggttga

SEQ ID NO: 90 - DNA, 55 bp, Artificial
  Aptamer
gtcaatgccaggtctcggacctggaatccgccacccgcatagaaccagggttgac

SEQ ID NO: 91 - DNA, 53 bp, Artificial
  Aptamer
gtcaatgccagtcggacctggaatccgccacccgcattagaaccagggttgac

SEQ ID NO: 92 - DNA, 55 bp, Artificial
  Aptamer
gtcaatgccaggtctcggacctggaatccgccacccgcttagaaccagggttgac

SEQ ID NO: 93 - DNA, 52 bp, Artificial
  Aptamer
gtcaatgccagcggacctggaatccgccacccgcattagaaccagggttgac

SEQ ID NO: 94 - DNA, 40 bp, Artificial
  central region
ggacctggaatccgccacccgcattagaaccagggttgac

SEQ ID NO: 95 - DNA, 40 bp, Artificial
  central region
aactttcgcgtgtggttggtagggctaggtgtatacgcat